(19) United States
(12) Patent Application Publication
(10) Pub. No.: US 2008/0064730 A1
(43) Pub. Date: Mar. 13, 2008

(54) Azine Compounds for Combating Animal Pests

(75) Inventors: Michael Hofmann, Ludwigshafen (DE); Ernst Baumann, Dudenhofen (DE); Wolfgang von Deyn, Neustadt (DE); Markus Kordes, Frankenthal (DE); Livio Tedeschi, Mannheim (DE); Michael F. Treacy, Corpus Christi, TX (US); Deborah L. Culbertson, Fuquay Varina, NC (US); Toni Bucci, Fuquay Varina, NC (US); Hong-Ming Shieh, Newtown, PA (US); Robert Arotin, Levittown, PA (US); Nicole Johnson, Cream Ridge, NJ (US)

Correspondence Address: HUTCHISON LAW GROUP PLLC, PO BOX 31686, RALEIGH, NC, 27612, US

(73) Assignee: BASF Aktiengesellschaft, Ludwigshafen (DE)

(21) Appl. No.: 11/791,424

(22) PCT Filed: Nov. 25, 2005
(86) PCT No.: PCT/EP05/12636

§ 371 Date: May 23, 2007

Related U.S. Application Data
(60) Provisional application No. 60/631,198, filed on Nov. 26, 2004.

(51) Int. Cl. A01N 43/10; A01N 43/12; A01N 43/40; A01P 5/00; A01P 7/04; C07D 213/60; C07D 333/22; C07D 333/38; C07D 333/54

(52) U.S. Cl. 514/357; 514/438; 514/444; 546/329; 549/60; 549/74

(57) ABSTRACT

The present invention relates to new azine compounds which are useful combating animal pests, in particular insects and nematodes and to the salts thereof. The invention also relates to a method for combating insects, nematodes and arachnids. The azine compounds of the invention are described by the general formula (I) wherein . . . is absent or a covalent bond; n is 0 or 1, in particular 0; A is an optionally substituted cyclic radical selected from phenyl and a 5- or 6-membered heterocyclic radical with 1, 2, 3, or 4 heteroatoms; Ar is an optionally substituted aromatic radical selected from phenyl, pyridyl, pyrimidyl, furyl and thienyl, x is selected from halogen, OR⁷, SR⁷, SOR⁷, SO₂R⁷, C₁-C₄-alkyl and C₁-C₄-haloalkyl; and wherein R¹ to R⁴ and R⁷ are as described in the claims and the specification.

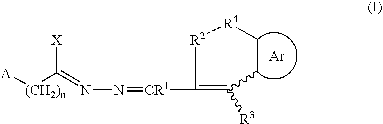

The present invention relates to new azine compounds which are useful for combating animal pests, in particular insects and nematodes. The invention also relates to a method for combating insects, nematodes and arachnids.

In spite of commercial pesticides available today, damage to crops, both growing and harvested, the damage of non-living material, in particular cellulose based materials such as wood or paper, and other nuisance, such as transmission of diseases, caused by animal pests still occur.

JP 2000169461 describes inter alia thiadiazolylcarbonylhydrazones of phenylketones having insecticidal or fungicidal activity. However, the insecticidal activity of these compounds is not satisfactory.

A. M. Islam et al., Egyptian Journal of Chemistry 1986, 29(4) p. 405-431 (CASREACT 111:173716) discloses several naphthalin-2-yl sulfonylhydrazones of aromatic aldehydes, which were screened against cotton leaf worm (Spodoptera literalis). However, the activity of these compounds against other pests is not satisfactory.

JP 2001172217 discloses ethylene derivatives having acaricidal activity of the formulae 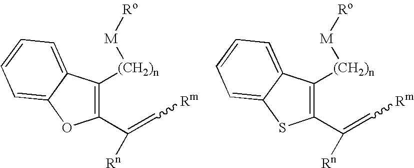
wherein n is 0, 1, or 2, Rⁿ and R^{o} are each a (substituted) aromatic radical or a (substituted) heterocyclic group, R^{m} is hydrogen, halogen, alkyl or the like and M is (inter alia) CH═N—N═C(CH₃). However, the acaricidal activity of these compounds is not satisfactory.

Therefore, there is continuing need to provide compounds which are useful for combating pests such as insects, nematodes and arachnids.

International application PCT/EP 2004/005681 discloses compounds of the general formula 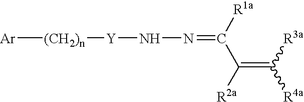
wherein Ar is an optionally substituted cyclic radical, selected from phenyl, naphthyl and heterocyclic radicals, n is 0 or 1, Y is inter alia CO or SO₂, R^{1a} is H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₂-C₁₀-haloalkynyl or optionally substituted phenyl, R^{2b} and R^{3a} are inter alia H, C₁-C₁₀-alkyl, C₁-C₁₀-haloalkyl, halogen, optionally substituted phenyl or cyano and R^{4a} is inter alia an optionally substituted aromatic radical selected from phenyl, pyridyl, pyrimidyl, furyl and thienyl. These compounds are active against insects and arachnids.

It is an object of the present invention to provide further compounds having a good activity against insects, nematodes and/or arachnids and thus are useful for combating said pests.

The inventors of the present application surprisingly found that this object is achieved by compounds of formula I as defined below and the salts thereof.

Therefore, the present invention relates to compounds of the general formula I 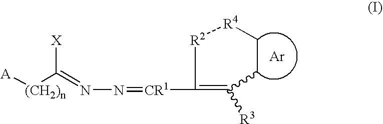
and to the salts thereof, wherein
. . . is absent or a covalent bond;
n is 0 or 1, in particular 0;
A is a cyclic radical selected from phenyl and a 5- or 6-membered heterocyclic radical with 1, 2, 3 or 4 heteroatoms which are selected, independently of one another, from O, N and S, and where the cyclic radical may have 1, 2, 3, 4 or 5 substituents R^{a} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁵R⁶, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, R⁵R⁶N—CO—, phenyl, benzyl and phenoxy, wherein phenyl, benzyl and phenoxy may be substituted by 1, 2, 3, 4 or 5 substituents R^{b} which are selected, independently of one another, from halogen, cyano, nitro, —C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁵R⁶, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl and R⁵R⁶N—CO—;
Ar is an aromatic radical selected from phenyl, pyridyl, pyrimidyl, furyl and thienyl, where the aromatic radical may carry 1 to 5 substituents R^{c} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, R⁵R⁶N—CO—, phenyl and phenoxy, wherein phenyl and phenoxy may be unsubstituted or substituted by 1, 2, 3, 4 or 5 substituents R^{b} as defined above;
X is selected from halogen, OR⁷, SR⁷, SOR⁷, SO₂R⁷, C₁-C₄-alkyl and C₁-C₄-haloalkyl;
R¹ is H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₂-C₁₀-haloalkynyl or phenyl which may be substituted by 1, 2, 3, 4 or 5 substituents R^{d} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁵R⁶, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl and R⁵R⁶N—CO—;
R² is a monovalent radical selected from H, halogen, cyano, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, hydroxy-C₁₋₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl and phenyl which may be substituted by 1, 2, 3, 4 or 5 substituents R^{b} as defined above;
R³ is H, halogen, cyano, C₁-C₁₀-alkyl C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₁-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl or phenyl which may be substituted by 1 to 5 substituents R^{b} as defined above;
R⁴ is hydrogen or has one of the meanings given for R^{c} or
   R⁴ together with R² is a bivalent radical, which is selected from O, S, N—R⁸, CR⁹═N, CH₂—CH₂, O—C(O) or O—CH₂;
R⁵ and R⁶, independently of one another, are H or C₁-C₁₀-alkyl;
R⁷ is selected from C₁-C₁₀-alkyl, C₁-C₁₀-alkylcarbonyl, C₁-C₁₀-haloalkyl, C₂-C₁₀-alkenyl, C₂-C₁₀-haloalkenyl, C₃-C₁₀-cycloalkyl and C₃-C₁₀-halocycloalkyl;
R⁸ is hydrogen, cyano, C₁-C₁₀-alkyl, C₁-C₁₀-haloalkyl, C₃-C₁₀-cycloalkyl, C₃-C₁₀-halocycloalkyl, C₁₋₁₀-alkenyl, C₂-C₁₀-haloalkenyl, C₂-C₁₀-alkynyl, C₃-C₁₀-haloalkynyl, C₁-C₁₀-haloalkylsulfonyl, O₁—C₁₋₁₀-alkylcarbonyl, C₁-C₁₀-haloalkylcarbonyl, R⁵R⁶N—CO—, phenyl or benzyl, wherein phenyl and benzyl may be substituted by 1, 2, 3, 4 or 5 substituents R^{b}; and
R⁹ is hydrogen or has one of the meanings given for R^{c}.

Due to their excellent activity, the compounds of the general formula I can be used for controlling pests, selected from harmful insects, arachnids and nematodes. The compounds of the formula I are in particular useful from combating insects and nematodes.

Accordingly, the invention further provides compositions for combating such pests, preferably in the form of directly sprayable-solutions, emulsions, pastes, oil dispersions, powders, materials for scattering, dusts or in the form of granules, which comprises a pesticidally effective amount of at least one compound of the general formula I or at least a salt thereof and at least one carrier which may be liquid and/or solid and which is preferably agronomically acceptable, and/or at least one surfactant.

Furthermore, the invention provides a method for combating such pests, which comprises contacting said pests, their habitat, breeding ground, food supply, plant, seed, soil, area, material or environment in which the animal pests are growing or may grow, or the materials, plants, seeds, soils, surfaces or spaces to be protected from an attack of or infestation by said pest, with a pesticidally effective amount of a compound of the general formula I as defined herein or a salt thereof.

The invention provides in particular a method for protecting crops, including seeds, from attack or infestation by harmful insects, arachnids and/or nematodes, said method comprises contacting a crop with a pesticidally effective amount of at least one compound of formula I as defined herein or with a salt thereof.

The invention also provides a method for protecting non-living materials from attack or infestation by the aforementioned pests, which method comprises contacting the non-living material with a pesticidally effective amount of at least one compound of formula I as defined herein or with a salt thereof.

Suitable compounds of the general formula I encompass all possible stereoisomers (cis/trans isomers) which may occur and mixtures thereof. Stereoisomeric centers are e.g. the carbon atom of the azine group (C═N—N═C) and the carbon atoms carrying the radicals R² and R³. The compounds of the general formula I may also have one or more centers of chirality, in which case they are present as mixtures of enantiomers or diastereomers. The present invention provides both the pure enantiomers or diastereomers or mixtures thereof. The compounds of the general formula I may also exist in the form of different tautomers if A or Ar carry amino or hydroxy groups. The invention comprises the single tautomers, if separable, as well as the tautomer mixtures.

Salts of the compounds of the formula I are preferably agriculturally acceptable salts. They can be formed in a customary method, e.g. by reacting the compound with an acid of the anion in question if the compound of formula I has a basic functionality or by reacting an acidic compound of formula I with a suitable base.

Suitable agriculturally useful salts are especially the salts of those cations or the acid addition salts of those acids whose cations and anions, respectively, do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH₄⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethylammonium, bis(2-hydroxyethyl)ammonium, benzyltrimethylammonium and benzyltriethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting the compounds of the formulae Ia and Ib with an acid of the corresponding anion, preferably of hydrochloric acid, hydrobromic acid, sulfuric acid, phosphoric acid or nitric acid.

The organic moieties mentioned in the above definitions of the variables are—like the term halogen—collective terms for individual listings of the individual group members. The prefix Cₙ-Cₘ indicates in each case the possible number of carbon atoms in the group.

“Halogen” will be taken to mean fluoro, chloro, bromo and iodo.

The term “C₁-C₁₀-alkyl” as used herein (and also in C₁-C₁₀-alkylsulfinyl and C₁-C₁₀-alkylsulfonyl) refers to a branched or unbranched saturated hydrocarbon group having 1 to 10 carbon atoms, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, octyl, 2-ethylhexyl, nonyl and decyl and their isomers. C₁-C₄-alkyl means for example methyl, ethyl, propyl, 1-methylethyl, butyl, 1-methylpropyl, 2-methylpropyl or 1,1-dimethylethyl.

The term “C₁-C₁₀-haloalkyl” as used herein (and also in C₁-C₁₀-haloalkylsulfinyl and C₁-C₁₀-haloalkylsulfonyl) refers to a straight-chain or branched alkyl group having 1 to 10 carbon atoms (as mentioned above), where some or all of the hydrogen atoms in these groups may be replaced by halogen atoms as mentioned above, for example C₁-C₄-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and the like. The term C₁-C₁₀-haloalkyl in particular comprises C₁-C₂-fluoroalkyl, which is synonym with methyl or ethyl, wherein 1, 2, 3, 4 or 5 hydrogen atoms are substituted by fluorine atoms, such as fluoromethyl, difluoromethyl trifluoromethyl, 1-fluoroethyl, 2-fluoroethyl, 2,2-difluoroethyl, 2,2,2-trifluoroethyl and pentafluoromethyl.

Similarly, “C₁-C₁₀-alkoxy” and “C₁-C₁₀-alkylthio” refer to straight-chain or branched alkyl groups having 1 to 10 carbon atoms (as mentioned above) bonded through oxygen or sulfur linkages, respectively, at any bond in the alkyl group. Examples include C₁-C₄-alkoxy such as methoxy, ethoxy, propoxy, isopropoxy, butoxy, sec-butoxy, isobutoxy and tert-butoxy, further C₁-C₄-alkylthio such as methylthio, ethylthio, propylthio, isopropylthio, and n-butylthio.

Accordingly, the terms “C₁-C₁₀-haloalkoxy” and “C₁-C₁₀-haloalkylthio” refer to straight-chain or branched alkyl groups having 1 to 10 carbon atoms (as mentioned above) bonded through oxygen or sulfur linkages, respectively, at any bond in the alkyl group, where some or all of the hydrogen atoms in these groups may be replaced by halogen atoms as mentioned above, for example C₁-C₂-haloalkoxy, such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy and pentafluoroethoxy, further C₁-C₂-haloalkylthio, such as chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio and pentafluoroethylthio and the like. Similarly the terms C₁-C₂-fluoroalkoxy and C₁-C₂-fluoroalkylthio refer to C₁-C₂-fluoroalkyl which is bound to the remainder of the molecule via an oxygen atom or a sulfur atom, respectively.

The term “C₂-C₁₀-alkenyl” as used herein intends a branched or unbranched unsaturated hydrocarbon group having 2 to 10 carbon atoms and a double bond in any position, such as ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

The term “C₂-C₁₀-haloalkenyl” as used herein intends a branched or unbranched unsaturated hydrocarbon group having 2 to 10 carbon atoms and a double bond in any position, where some or all of the hydrogen atoms in these groups may be replaced by halogen atoms as mentioned above.

Similarly, the term “C₂-C₁₀-alkenyloxy” as used herein intends a branched or unbranched unsaturated hydrocarbon group having 2 to 10 carbon atoms and a double bond in any position, the alkenyl group being bonded through oxygen linkages, respectively, at any bond in the alkenyl group, for example ethenyloxy, propenyloxy and the like.

Accordingly, the term “C₂-C₁₀-haloalkenyloxy” as used herein intends a branched or unbranched unsaturated hydrocarbon group having 2 to 10 carbon atoms and a double bond in any position, the alkenyl group being bonded through oxygen linkages, respectively, at any bond in the alkenyl group, where some or all of the hydrogen atoms in these groups may be replaced by halogen atoms as mentioned above.

The term “C₂-C₁₀-alkynyl” as used herein refers to a branched or unbranched unsaturated hydrocarbon group having 2 to 10 carbon atoms and containing at least one triple bond, such as ethynyl, propynyl, 1-butynyl, 2-butynyl, and the like.

The term “C₃-C₁₀-haloalkynyl” as used herein refers to a branched or unbranched unsaturated hydrocarbon group having 3 to 10 carbon atoms and containing at least one triple bond, where some or all of the hydrogen atoms in these groups may be replaced by halogen atoms as mentioned above, with the proviso that the halogen atom is not directly bound to the triple bond.

The term “C₂-C₁₀-alkynyloxy” as used herein refers to a branched or unbranched unsaturated hydrocarbon group having 2 to 10 carbon atoms and containing at least one triple bond, the alkynyl group being bonded through oxygen linkages at any bond in the alkynyl group.

Similarly, the term “C₃-C₁₀-haloalkynyloxy” as used herein refers to a branched or unbranched unsaturated hydrocarbon group having 3 to 10 carbon atoms and containing at least one triple bond, the group being bonded through oxygen linkages at any bond in the alkynyl group, where some or all of the hydrogen atoms in these group may be replaced by halogen atoms as mentioned above, with the proviso that the halogen atom is not directly bound to the triple bond.

The term “C₃-C₁₀-cycloalkyl” as used herein refers to a monocyclic 3- to 10-membered saturated carbon atom ring, e.g. cyclopropyl, cyclobutyl, cyclopentyl, cyclohexyl, cycloheptyl, cyclooctyl and cyclodecyl.

The term “C₃-C₁₀-halocycloalkyl” as used herein refers to a monocyclic 3- to 10-membered saturated carbon atom ring, e.g. cyclopropyl, cyclobutyl, cyclopentyl, cyclohexyl, cycloheptyl, cyclooctyl and cyclodecyl, where some or all of the hydrogen atoms in these groups may be replaced by halogen atoms as mentioned above, for example chloro-, dichloro- and trichlorocyclopropyl, fluoro-, difluoro- and trifluorocyclopropyl, chloro-, dichloro-, trichloro-, tetrachloro-, pentachloro- and hexachlorocyclohexyl and the like.

The term “C₁-C₁₀-alkylcarbonyl” as used herein refers to C₁-C₁₀-alkyl which is bound to the remainder of the molecule via a carbonyl group. Examples include CO—CH₃, CO—C₂H₅, CO—CH₂—C₂H₅, CO—CH(CH₃)₂, n-butylcarbonyl, CO—CH(CH₃)—C₂H₅, CO—CH₂—CH(CH₃)₂, CO—C(CH₃)₃, n-pentylcarbonyl, 1-methylbutylcarbonyl, 2-methylbutylcarbonyl, 3-methylbutylcarbonyl, 2,2-dimethylpropylcarbonyl, 1-ethylpropylcarbonyl, n-hexylcarbonyl, 1,1-dimethylpropylcarbonyl, 1,2-dimethylpropylcarbonyl, 1-methylpentylcarbonyl, 2-methylpentylcarbonyl, 3-methylpentylcarbonyl, 4-methylpentylcarbonyl, 1,1-dimethylbutylcarbonyl, 1,2-dimethylbutylcarbonyl, 1,3-dimethylbutylcarbonyl, 2,2-dimethylbutylcarbonyl, 2,3-dimethylbutylcarbonyl, 3,3-dimethylbutylcarbonyl, 1-ethylbutylcarbonyl, 2-ethylbutylcarbonyl, 1,1,2-trimethylpropylcarbonyl, 1,2,2-trimethylpropylcarbonyl, 1-ethyl-1-methylpropylcarbonyl or 1-ethyl-2-methylpropylcarbonyl.

The term “C₁-C₁₀-alkoxycarbonyl” as used herein refers to C₁-C₁₀-alkoxy which is bound to the remainder of the molecule via a carbonyl group. Examples include CO—OCH₃, CO—OC₂H₅, CO—OCH₂—C₂Hs, COOCH(CH₃)₂, n-butoxycarbonyl, CO—OCH(CH₃)—C₂H₅, CO—OCH₂—CH(CH₃)₂, CO—OC(CH₃)₃, n-pentoxycarbonyl, 1-methylbutoxycarbonyl, 2-methylbutoxycarbonyl, 3-methylbutoxycarbonyl, 2,2-dimethylpropoxycarbonyl, 1-ethylpropoxycarbonyl, n-hexoxycarbonyl, 1,1-dimethylpropoxycarbonyl, 1,2-dimethylpropoxycarbonyl, 1-methylpentoxycarbonyl, 2-methylpentoxycarbonyl, 3-methylpentoxycarbonyl, 4-methylpentoxycarbonyl, 1,1-dimethylbutoxycarbonyl, 1,2-dimethylbutoxycarbonyl, 1,3-dimethylbutoxycarbonyl, 2,2-dimethylbutoxycarbonyl, 2,3-dimethylbutoxycarbonyl, 3,3-dimethylbutoxycarbonyl, 1-ethylbutoxycarbonyl, 2-ethylbutoxycarbonyl, 1,1,2-trimethylpropoxycarbonyl, 1,2,2-trimethylpropoxycarbonyl, 1-ethyl-1-methylpropoxycarbonyl or 1-ethyl-2-methylpropoxycarbonyl.

The term “halo-C₁-C₁₀-alkoxycarbonyl” as used herein refers to C₁-C₁₀-haloalkoxy which is bound to the remainder of the molecule via a carbonyl group.

The terms “hydroxy-C₁-C₁₀-alkyl”, “C₁-C₁₀-alkoxy-C₁-C₁₀-alkyl”, “halo-C₁-C₁₀-alkoxy-C₁-C₁₀-alkyl”, “C₁-C₁₀-alkoxycarbonyl-C₁-C₁₀-alkyl”, “halo-C₁-C₁₀-alkoxycarbonyl-C₁-C₁₀-alkyl” as used herein, refer to C₁-C₁₀-alkyl, as defined herein, in particular to methyl, ethyl, 1-propyl or 2-propyl, which is substituted by one radical selected from hydroxy, C₁-C₁₀-alkoxy, C₁-C₁₀-haloalkoxy, C₁-C₁₀-alkoxycarbonyl or C₁-C₁₀-haloalkoxycarbonyl.

The term “5- or 6-membered heterocyclic radical with 1, 2, 3 or 4 heteroatoms which are selected, independently of one another, from O, N and S” comprises monocyclic 5 or 6-membered heteroaromatic rings and nonaromatic saturated or partially unsaturated 5- or 6-membered mono-heterocycles, which carry 1, 2, 3, or 4 heteroatoms as ring members. The heterocyclic radical may be attached to the remainder of the molecule via a carbon ring member or via a nitrogen ring member.

Examples for non-aromatic rings include pyrrolidinyl, pyrazolinyl, imidazolinyl, pyrrolinyl, pyrazolinyl, imidazolinyl, tetrahydrofuranyl, dihydrofuranyl, 1,3-dioxolanyl, dioxolenyl, thiolanyl, dihydrothienyl, oxazolidinyl, isoxazolidinyl, oxazolinyl, isoxazolinyl, thiazolinyl, isothiazolinyl, thiazolidinyl, isothiazolidinyl, oxathiolanyl, piperidinyl, piperazinyl, pyranyl, dihydropyranyl, tetrahydropyranyl, dioxanyl, thiopyranyl, dihydrothiopyranyl, tetrahydrothiopyranyl, morpholinyl, thiazinyl and the like.

Examples for monocyclic 5- to 6-membered heteroaromatic rings include triazinyl, pyrazinyl, pyrimidyl, pyridazinyl, pyridyl, thienyl, furyl, pyrrolyl, pyrazolyl, imidazolyl, triazolyl, tetrazolyl, thiazolyl, oxazolyl, thiadiazolyl, oxadiazolyl, isothiazolyl and isoxazolyl.

With respect to the use according to the invention of the compounds of formula I, particular preference is given to the following meanings of the substituents, in each case on their own or in combination:

Preference is given to compounds of formula I, wherein A in formula I is a cyclic radical selected from phenyl, thienyl, furyl, pyrrolyl, pyrazolyl, imidazolyl, oxazolyl, isoxazolyl, thiazolyl, isothiazoloyl, pyridyl, pyrimidinyl, pyrazinyl, and pyridazinyl and where the cyclic radical may be unsubstituted or substituted as described above. In particular the aforementioned radicals are unsubstituted or substituted by 1, 2 or 3 radicals R^{a} as defined above.

Preferred radicals R^{a} comprise halogen, CN, C₁-C₄-alkyl, C₁-C₄-alkoxy, C₁-C₄-haloalkoxy and C₁-C₄-haloalkyl, in particular F, Cl, methyl, methoxy, difluoromethyl, trifluoromethyl, trifluoromethoxy and difluoromethoxy.

More preference is given to compounds of formula I, wherein A is a cyclic radical selected from phenyl, thienyl, and pyridyl, where the cyclic radical may be substituted by 1, 2 or 3 substituents R^{a} which are as defined above and which are preferably selected, independently of one another, from halogen, C₁-C₄-alkyl, C₁-C₄-alkoxy, C₁-C₄-haloalkoxy and C₁-C₄-haloalkyl, in particular from F, Cl, methyl, methoxy, difluoromethyl, trifluoromethyl, trifluoromethoxy and difluoromethoxy. Examples of preferred radicals A comprise 2-thienyl, 3-bromothien-2-yl, 4-bromothien-2-yl, 5-bromothien-2-yl, 4,5-dibromothien-2-yl, 3-chlorothien-2-yl, 4-chlorothien-2-yl, 5-chlorothien-2-yl, 3-chloro-4-methylthien-2-yl, 3-methylthien-2-yl, 4-methylthien-2-yl, 5-methylthien-2-yl, pyridin-2-yl, pyridin-3-yl, 6-chloropyrid-3-yl, 6-bromopyrid-3-yl, 6-methylthiopyrid-2-yl, 6-methylpyrid-2-yl, 3-fluoropyrid-2-yl, 6-bromopyrid-2-yl, pyridin-4-yl, phenyl and 2-fluorophenyl.

A very preferred embodiment of the invention relates to compounds of the formula I, wherein A is thienyl, in particular 2-thienyl which is unsubstituted or substituted by 1, 2 or 3 radicals R^{a} as defined above, the radicals R^{a} being preferably selected, independently of one another, from halogen, C₁-C₄-alkyl, C₁-C₄-alkoxy, C₁-C₄-haloalkoxy and C₁-C₄-haloalkyl, in particular from F, Cl, methyl, methoxy, difluoromethyl, trifluoromethyl, trifluoromethoxy and difluoromethoxy.

Another very preferred embodiment of the invention relates to compounds of the formula I, wherein A is pyridyl, in particular 2- or 3-pyridyl, more preferably 2-pyridyl which is unsubstituted or substituted by 1, 2 or 3 radicals R^{a} as defined above, the radicals R^{a} being preferably selected, independently of one another, from halogen, C₁-C₄-alkyl, C₁-C₄-alkoxy, C₁-C₄-haloalkoxy and C₁-C₄-haloalkyl, in particular from F, Cl, methyl, methoxy, difluoromethyl, trifluoromethyl, trifluoromethoxy and difluoromethoxy.

A further very preferred embodiment of the invention relates to compounds of the formula I, wherein A is phenyl, which is unsubstituted or substituted by 1, 2 or 3 radicals R^{a} as defined above, the radicals R^{a} being preferably selected, independently of one another, from halogen, C₁-C₄-alkyl, C₁-C₄-alkoxy, C₁-C₄-haloalkoxy and C₁-C₄-haloalkyl, in particular from F, Cl, methyl, methoxy, difluoromethyl, trifluoromethyl, trifluoromethoxy and difluoromethoxy.

Preference is given to compounds of the general formula I, wherein X in formula I is selected from Cl, Br, OR⁷, SR⁷, SO₂R⁷ and methyl, wherein R⁷ is as defined above. In particular R⁷ is selected from C₁-C₄-alkyl and C₁-C₂-fluoroalkyl. More preferably X is selected from Cl, OCH₃, OCHF₂, SCH₃, SO₂CH₃, SO₂CF₃, SO₂CH₂CF₃ and SCF₃.

R¹ is preferably hydrogen.

Ar is preferably phenyl, which is unsubstituted or substituted by 1, 2, 3 or 4, in particular 1, 2 or 3 radicals R^{c} as defined above. A skilled person will appreciate that in case of R⁴ being different from hydrogen, R⁴ is one of the 1 to 4 radicals R^{c} as defined above or R⁴ together with R² is the aforementioned bivalent radical.

Preferably the radicals R^{c} are selected, independently of one another, from halogen, CN, C₁-C₄alkyl, C₁-C₄-alkoxy, C₁-C₄-haloalkoxy and C₁-C₄-haloalkyl, more preferably F, Cl, CN, C₁-C₃-alkoxy, in particular methoxy, trifluoromethyl, difluoromethyl, trifluoromethoxy, difluoromethoxy and methyl.

Preference is also given to compounds of the formula I, wherein R³ is selected from hydrogen, halogen and C₁-C₄-alkyl, in particular hydrogen, fluorine, chlorine or methyl, more preferably hydrogen or methyl and especially hydrogen.

In a preferred embodiment of the invention the radical R² in formula I is a monovalent radical, i.e. R² and R⁴ together do not form a bivalent radical. In this embodiment R² is preferably selected from hydrogen, halogen, C₁-C₄-alkyl, C₁-C₄-alkoxy, C₁-C₄-haloalkoxy and C₁-C₄-haloalkyl. More preferably R² is hydrogen, fluorine, chlorine, bromine, methyl or ethyl. In this embodiment R⁴ is hydrogen or a radical R^{c} as defined above, in particular hydrogen. In this embodiment Ar in formula I is preferably phenyl, which is unsubstituted or substituted by 1, 2, 3 or 4, in particular 1, 2 or 3 radicals R^{c} as defined above.

Another embodiment of the invention relates to compounds of the formula I, wherein R⁴ together with R² is a bivalent radical as defined above and which is preferably selected from O, S, CH₂—CH₂ and O—C(O). More preferably R⁴ and R² together are an oxygen atom or O—C(O) in particular 0. In this embodiment Ar in formula I is preferably phenyl, which is unsubstituted or substituted by 1, 2 or 3, in particular 0, 1 or 2 radicals R^{c} as defined above.

Apart from that, R^{b} is preferably selected from halogen, C₁-C₄-alkyl, C₁-C₄-alkoxy, C₁-C₄-haloalkoxy and C₁-C₄-haloalkyl.

A very preferred embodiment of the invention relates to compounds of the general formula Ia: 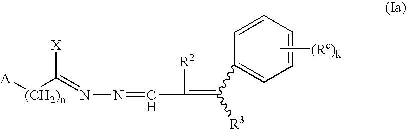
wherein k is 0, 1, 2 or 3, and wherein A, n, X, R², R³ and R¹ are as defined above.

Amongst the compounds Ia those are preferred, wherein n is 0 and wherein A, n, X, R², R³ and R^{c} have the meanings given as preferred.

Examples of compounds Ia are given in the following tables 1 to 90

Table 1:

Compounds of the formula Ia, wherein n is 0 and R³ is H, R² is H and k is 0 (i.e. (R^{c})ₖ is absent) and wherein X and A are given in table A;

Table 2:

Compounds of the formula Ia, wherein n is 0 and R³ is H, R² is H and (R^{c})ₖ is 4-fluoro and wherein X and A are given in table A;

Table 3:

Compounds of the formula Ia, wherein n is 0 and R³ is H, R² is —H and (R^{c})ₖ is 4-chloro and wherein X and A are given table A;

Table 4:

Compounds of the formula Ia, wherein n is 0 and R³ is H, R² is H and (R^{k} is 3-fluoro and wherein X and A are given in table A;

Table 5:

Compounds of the formula Ia, wherein n is 0 and R³ is H, R² is H and (R^{c})ₖ is 3-difluoromethoxy and wherein X and A are given in table A;

Table 6:

Compounds of the formula Ia, wherein n is 0 and R³ is H, R² is H and (R^{c})ₖ is 3-trifluoromethyl and wherein X and A are given in table A;

Table 7:

Compounds of the formula Ia, wherein n is 0 and R³ is H, R² is H and (R^{c})ₖ is 4-fluoro-3-trifluoromethyl and wherein X and A are given in table A;

Table 8:

Compounds of the formula Ia, wherein n is 0 and R³ is H, R² is H and (R^{c})ₖ is 4-methoxy-3-trifluoromethyl and wherein X and A are in given table A;

Table 9:

Compounds of the formula Ia, wherein n is 0 and R³ is H, R² is H and (R^{c})ₖ is 4-methylthio-3-trifluoromethyl and wherein X and A are given in table A;

Table 10:

Compounds of the formula Ia, wherein n is 0 and R³ is H, R² is H and (R^{c})ₖ is 2-F and wherein X and A are given in table A;

Table 11:

Compounds of the formula Ia, wherein n is 0 and R³ is H, R² is H and (R^{c})ₖ is 2-CH₃ and wherein X and A are given in table A;

Table 12:

Compounds of the formula Ia, wherein n is 0 and R³ is H, R² is H and (R^{c})ₖ is 2-OCH₃ and wherein X and A are given in table A;

Table 13:

Compounds of the formula Ia, wherein n is 0 and R³ is H, R² is H and (R^{c})ₖ is 2-CF₃ and wherein X and A are given in table A;

Table 14:

Compounds of the formula Ia, wherein n is 0 and R³ is H, R² is H and (R^{c})ₖ is 4-OCH₃ and wherein X and A are given in table A;

Table 15:

Compounds of the formula Ia, wherein n is 0 and R³ is H, R² is H and (R^{c})ₖ is 2-OCHF₂ and wherein X and A are given in table A;

Table 16:

Compounds of the formula Ia, wherein n is 0 and R³ is H, R² is F and k is 0 (i.e. (R^{c})ₖ is absent) and wherein X and A are given in table A;

Table 17:

Compounds of the formula Ia, wherein n is 0 and R³ is H, R² is F and (R^{c})ₖ is 4-fluoro and wherein X and A are given in table A;

Table 18:

Compounds of the formula Ia, wherein n is 0 and R³ is H, R² is F and (R^{c})ₖ is 4-chloro and wherein X and A are given in table A;

Table 19:

Compounds of the formula Ia, wherein n is 0 and R³ is H, R² is F and (R^{c})ₖ is 3-fluoro and wherein X and A are given in table A;

Table 20:

Compounds of the formula Ia, wherein n is 0 and R³ is H, R² is F and (R^{c})ₖ is 3-difluoromethoxy and wherein X and A are given in table A;

Table 21:

Compounds of the formula Ia, wherein n is 0 and R³ is H, R² is F and (R)ₖ is 3-trifluoromethyl and wherein X and A are given in table A;

Table 22:

Compounds of the formula Ia, wherein n is 0 and R³ is H, R² is F and (R^{c})ₖ is 4-fluoro-3-trifluoromethyl and wherein X and A are given in table A;

Table 23:

Compounds of the formula Ia, wherein n is 0 and R³ is H, R² is F and (R^{c})ₖ is 4-methoxy-3-trifluoromethyl and wherein X and A are given in table A;

Table 24:

Compounds of the formula Ia, wherein n is 0 and R³ is H, R² is F and (R)ₖ is 4-methylthio-3-trifluoromethyl and wherein X and A are given in table A;

Table 25:

Compounds of the formula Ia, wherein n is 0 and R³ is H, R² is F and (R^{c})ₖ is 2-F and wherein X and A are given in table A;

Table 26:

Compounds of the formula Ia, wherein n is 0 and R³ is H, R² is F and (R^{c})ₖ is 2-CH₃ and wherein X and A are given in table A;

Table 27:

Compounds of the formula Ia, wherein n is 0 and R³ is H, R² is F and (R^{c})ₖ is 2-OCH₃ and wherein X and A are given in table A;

Table 28:

Compounds of the formula Ia, wherein n is 0 and R³ is H, R² is F and (R^{c})ₖ is 2-CF₃ and wherein X and A are given in table A;

Table 29:

Compounds of the formula Ia, wherein n is 0 and R³ is H, R² is F and (R^{c})ₖ is 4-OCH₃ and wherein X and A are given in table A;

Table 30:

Compounds of the formula Ia, wherein n is 0 and R³ is H, R² is F and (R^{c})ₖ is 2-OCHF₂ and wherein X and A are given in table A;

Table 31:

Compounds of the formula Ia, wherein n is 0 and R³ is H, R² is Cl and k is 0 (i.e. (R^{c})ₖ is absent) and wherein X and A are given in table A;

Table 32:

Compounds of the formula Ia, wherein n is 0 and R³ is H, R² is Cl and (R^{c})ₖ is 4-fluoro and wherein X and A are given in table A;

Table 33:

Compounds of the formula Ia, wherein n is 0 and R³ is H, R² is Cl and (R^{c})ₖ is 4-chloro and wherein X and A are given in table A;

Table 34:

Compounds of the formula Ia, wherein n is 0 and R³ is H, R² is Cl and (R^{c})ₖ is 3-fluoro and wherein X and A are given in table A;

Table 35:

Compounds of the formula Ia, wherein n is 0 and R³ is H, R² is Cl and (R^{c})ₖ is 3-difluoromethoxy and wherein X and A are given in table A;

Table 36:

Compounds of the formula Ia, wherein n is 0 and R³ is H, R² is Cl and (R^{c})ₖ is 3-trifluoromethyl and wherein X and A are given in table A;

Table 37:

Compounds of the formula Ia, wherein n is 0 and R³ is H, R² is Cl and (R^{c})ₖ is 4-fluoro-3-trifluoromethyl and wherein X and A are given in table A;

Table 38:

Compounds of the formula Ia, wherein n is 0 and R³ is H, R² is Cl and (R^{c})ₖ is 4-methoxy-3-trifluoromethyl and wherein X and A are given in table A;

Table 39:

Compounds of the formula Ia, wherein n is 0 and R³ is H, R² is Cl and (R^{c})ₖ is 4-methylthio-3-trifluoromethyl and wherein X and A are given in table A;

Table 40:

Compounds of the formula Ia, wherein n is 0 and R³ is H, R² is Cl and (R^{c})ₖ is 2-F and wherein X and A are given in table A;

Table 41:

Compounds of the formula Ia, wherein n is 0 and R³ is H, R² is Cl and (R^{c})ₖ is 2-CH₃ and wherein X and A are given in table A;

Table 42:

Compounds of the formula Ia, wherein n is 0 and R³ is H, R² is Cl and (R^{c})ₖ is 2-OCH₃ and wherein X and A are given in table A;

Table 43:

Compounds of the formula Ia, wherein n is 0 and R³ is H, R² is Cl and (R^{c})ₖ is 2-CF₃ and wherein X and A are given in table A;

Table 44:

Compounds of the formula Ia, wherein n is 0 and R³ is H, R² is Cl and (R^{c})ₖ is 4-OCH₃ and wherein X and A are given in table A;

Table 45:

Compounds of the formula Ia, wherein n is 0 and R³ is H, R² is Cl and (R^{c})ₖ is 2-OCHF₂ and wherein X and A are given in table A;

Table 46:

Compounds of the formula Ia, wherein n is 0 and R³ is H, R² is Br and k is 0 (i.e. (R^{c})ₖ is absent) and wherein X and A are given in table A;

Table 47:

Compounds of the formula Ia, wherein n is 0 and R³ is H, R² is Br and (R^{c})ₖ is 4-fluoro and wherein X and A are given in table A;

Table 48:

Compounds of the formula Ia, wherein n is 0 and R³ is H, R² is Br and (R^{c})ₖ is 4-chloro and wherein X and A are given in table A;

Table 49:

Compounds of the formula Ia, wherein n is 0 and R³ is H, R² is Br and (R^{c})ₖ is 3-fluoro and wherein X and A are given in table A;

Table 50:

Compounds of the formula Ia, wherein n is 0 and R³ is H, R² is Br and (R^{c})ₖ is 3-difluoromethoxy and wherein X and A are given in table A;

Table 51:

Compounds of the formula Ia, wherein n is 0 and R³ is H, R² is Br and (R^{c})ₖ is 3-trifluoromethyl and wherein X and A are given in table A;

Table 52:

Compounds of the formula Ia, wherein n is 0 and R³ is H, R² is Br and (R^{c})ₖ is 4-fluoro-3-trifluoromethyl and wherein X and A are given in table A;

Table 53:

Compounds of the formula Ia, wherein n is 0 and R³ is H, R² is Br and (R^{c})ₖ is 4-methoxy-3-trifluoromethyl and wherein X and A are given in table A;

Table 54:

Compounds of the formula Ia, wherein n is 0 and R³ is H, R² is Br and (R^{c})ₖ is 4-methylthio-3-trifluoromethyl and wherein X and A are given in table A;

Table 55:

Compounds of the formula Ia, wherein n is 0 and R³ is H, R² is Br and (R^{c})ₖ is 2-F and wherein X and A are given in table A;

Table 56:

Compounds of the formula Ia, wherein n is 0 and R³ is H, R² is Br and (R^{c})ₖ is 2-CH₃ and wherein X and A are given in table A;

Table 57:

Compounds of the formula Ia, wherein n is 0 and R³ is H, R² is Br and (R^{c})ₖ is 2-OCH₃ and wherein X and A are given in table A;

Table 58:

Compounds of the formula Ia, wherein n is 0 and R³ is H, R² is Br and (R^{c})ₖ is 2-CF₃ and wherein X and A are given in table A;

Table 59:

Compounds of the formula Ia, wherein n is 0 and R³ is H, R² is Br and (R^{c})ₖ is 4-OCH₃ and wherein X and A are given in table A;

Table 60:

Compounds of the formula Ia, wherein n is 0 and R³ is H, R² is Br and (R^{c})ₖ is 2-OCHF₂ and wherein X and A are given in table A;

Table 61:

Compounds of the formula Ia, wherein n is 0 and R³ is H, R² is CH₃ and k is 0 (i.e. (R^{c})ₖ is absent) and wherein X and A are given in table A;

Table 62:

Compounds of the formula Ia, wherein n is 0 and R³ is H, R² is CH₃ and (R^{c})ₖ is 4-fluoro and wherein X and A are given in table A;

Table 63:

Compounds of the formula Ia, wherein n is 0 and R³ is H, R² is CH₃ and (R^{c})ₖ is 4-chloro and wherein X and A are given in table A;

Table 64:

Compounds of the formula Ia, wherein n is 0 and R³ is H, R² is CH₃ and (R^{c})ₖ is 3-fluoro and wherein X and A are given in table A;

Table 65:

Compounds of the formula Ia, wherein n is 0 and R³ is H, R² is CH₃ and (R^{c})ₖ is 3-difluoromethoxy and wherein X and A are given in table A;

Table 66:

Compounds of the formula Ia, wherein n is 0 and R³ is H, R² is CH₃ and (R^{c})ₖ is 3-trifluoromethyl and wherein X and A are given in table A;

Table 67:

Compounds of the formula Ia, wherein n is 0 and R³ is H, R² is CH₃ and (R^{c})ₖ is 4-fluoro-3-trifluoromethyl and wherein X and A are given in table A;

Table 68:

Compounds of the formula Ia, wherein n is 0 and R³ is H, R² is CH₃ and (R^{c})ₖ is 4-methoxy-3-trifluoromethyl and wherein X and A are given in table A; and

Table 69:

Compounds of the formula Ia, wherein n is 0 and R³ is H, R² is CH₃ and (R^{c})ₖ is 4-methylthio-3-trifluoromethyl and wherein X and A are in given table A.

Table 70:

Compounds of the formula Ia, wherein n is 0 and R³ is H, R² is CH₃ and (R^{c})ₖ is 2-F and wherein X and A are given in table A;

Table 71:

Compounds of the formula Ia, wherein n is 0 and R³ is H, R² is CH₃ and (R^{c})ₖ is 2-CH₃ and wherein X and A are given in table A;

Table 72:

Compounds of the formula Ia, wherein n is 0 and R³ is H, R² is CH₃ and (R^{c})ₖ is 2-OCH₃ and wherein X and A are given in table A;

Table 73:

Compounds of the formula Ia, wherein n is 0 and R³ is H, R² is CH₃ and (R^{c})ₖ is 2-CF₃ and wherein X and A are given in table A;

Table 74:

Compounds of the formula Ia, wherein n is 0 and R³ is H, R² is CH₃ and (R^{c})ₖ is 4-OCH₃ and wherein X and A are given in table A;

Table 75:

Compounds of the formula Ia, wherein n is 0 and R³ is H, R² is CH₃ and (R^{c})ₖ is 2-OCHF₂ and wherein X and A are given in table A;

Table 76:

Compounds of the formula Ia, wherein n is 0 and R³ is H, R² is CH₂CH₃ and k is 0 (i.e. (R^{c})ₖ is absent) and wherein X and A are given in table A;

Table 77:

Compounds of the formula Ia, wherein n is 0 and R³ is H, R² is CH₂CH₃ and (R^{c})ₖ is 4-fluoro and wherein X and A are given in table A;

Table 78:

Compounds of the formula Ia, wherein n is 0 and R³ is H, R² is CH₂CH₃ and (R^{c})ₖ is 4-chloro and wherein X and A are given in table A;

Table 79:

Compounds of the formula Ia, wherein n is 0 and R³ is H, R² is CH₂CH₃ and (R^{c})ₖ is 3-fluoro and wherein X and A are given in table A;

Table 80:

Compounds of the formula Ia, wherein n is 0 and R³ is H, R² is CH₂CH₃ and (R^{c})ₖ is 3-difluoromethoxy and wherein X and A are given in table A;

Table 81:

Compounds of the formula Ia, wherein n is 0 and R³ is H, R² is CH₂CH₃ and (R^{c})ₖ is 3-trifluoromethyl and wherein X and A are given in table A;

Table 82:

Compounds of the formula Ia, wherein n is 0 and R³ is H, R² is CH₂CH₃ and (R^{c})ₖ is 4-fluoro-3-trifluoromethyl and wherein X and A are given in table A;

Table 83:

Compounds of the formula Ia, wherein n is 0 and R³ is H, R² is CH₂CH₃ and (R^{c})ₖ is 4-methoxy-3-trifluoromethyl and wherein X and A are given in table A;

Table 84:

Compounds of the formula Ia, wherein n is 0 and R³ is H, R² is CH₂CH₃ and (R^{c})ₖ is 4-methylthio-3-trifluoromethyl and wherein X and A are given in table A;

Table 85:

Compounds of the formula Ia, wherein n is 0 and R³ is H, R² is CH₂CH₃ and (R^{c})ₖ is 2-F and wherein X and A are given in table A;

Table 86:

Compounds of the formula Ia, wherein n is 0 and R³ is H, R² is CH₂CH₃ and (R^{c})ₖ is 2-CH₃ and wherein X and A are given in table A;

Table 87:

Compounds of the formula Ia, wherein n is 0 and R³ is H, R² is CH₂CH₃ and (R^{c})ₖ is 2-OCH₃ and wherein X and A are given in table A;

Table 88:

Compounds of the formula Ia, wherein n is 0 and R³ is H, R² is CH₂CH₃ and (R^{c})ₖ is 2-CF₃ and wherein X and A are given in table A;

Table 89:

Compounds of the formula Ia, wherein n is 0 and R^{a} is H, R² is CH₂CH₃ and (R^{c})ₖ is 4-OCH₃ and wherein X and A are given in table A;

Table 90:

Compounds of the formula Ia, wherein n is 0 and R³ is H, R² is CH₂CH₃ and (R^{c})ₖ is 2-OCHF₂ and wherein X and A are given in table A;

TABLE A

| | A | X |
|---|---|---|
| A-1 | 2-thienyl | Cl |
| A-2 | 3-thienyl | Cl |
| A-3 | 3-bromothien-2-yl | Cl |
| A-4 | 4-bromothien-2-yl | Cl |
| A-5 | 5-bromothien-2-yl | Cl |
| A-6 | 4,5-dibromothien-2-yl | Cl |
| A-7 | 3-chlorothien-2-yl | Cl |
| A-8 | 4-chlorothien-2-yl | Cl |
| A-9 | 5-chlorothien-2-yl | Cl |
| A-10 | 3-chloro-4-methylthien-2-yl | Cl |
| A-11 | 3-methylthien-2-yl | Cl |
| A-12 | 4-methylthien-2-yl | Cl |
| A-13 | 5-methylthien-2-yl | Cl |
| A-14 | pyridin-2-yl | Cl |
| A-15 | pyridin-3-yl | Cl |
| A-16 | 6-chloropyrid-3-yl | Cl |
| A-17 | 6-bromopyrid-3-yl | Cl |
| A-18 | 6-methylthiopyrid-2-yl | Cl |
| A-19 | 2-fluorophenyl | Cl |
| A-20 | phenyl | Cl |
| A-21 | 6-methylpyrid-2-yl | Cl |
| A-22 | 6-bromopyrid-2-yl | Cl |
| A-23 | 3-fluoropyrid-2-yl | Cl |
| A-24 | pyrid-4-yl | Cl |
| A-25 | 2-thienyl | CH₃ |
| A-26 | 3-thienyl | CH₃ |
| A-27 | 3-bromothien-2-yl | CH₃ |
| A-28 | 4-bromothien-2-yl | CH₃ |
| A-29 | 5-bromothien-2-yl | CH₃ |
| A-30 | 4,5-dibromothien-2-yl | CH₃ |
| A-31 | 3-chlorothien-2-yl | CH₃ |
| A-32 | 4-chlorothien-2-yl | CH₃ |
| A-33 | 5-chlorothien-2-yl | CH₃ |
| A-34 | 3-chloro-4-methylthien-2-yl | CH₃ |
| A-35 | 3-methylthien-2-yl | CH₃ |
| A-36 | 4-methylthien-2-yl | CH₃ |
| A-37 | 5-methylthien-2-yl | CH₃ |
| A-38 | pyridin-2-yl | CH₃ |
| A-39 | pyridin-3-yl | CH₃ |
| A-40 | 6-chloropyrid-3-yl | CH₃ |
| A-41 | 6-bromopyrid-3-yl | CH₃ |
| A-42 | 6-methylthiopyrid-2-yl | CH₃ |
| A-43 | 2-fluorophenyl | CH₃ |
| A-44 | phenyl | CH₃ |
| A-45 | 6-methylpyrid-2-yl | CH₃ |
| A-46 | 6-bromopyrid-2-yl | CH₃ |
| A-47 | 3-fluoropyrid-2-yl | CH₃ |
| A-48 | pyrid-4-yl | CH₃ |
| A-49 | 2-thienyl | OCH₃ |
| A-50 | 3-thienyl | OCH₃ |
| A-51 | 3-bromothien-2-yl | OCH₃ |
| A-52 | 4-bromothien-2-yl | OCH₃ |
| A-53 | 5-bromothien-2-yl | OCH₃ |
| A-54 | 4,5-dibromothien-2-yl | OCH₃ |
| A-55 | 3-chlorothien-2-yl | OCH₃ |
| A-56 | 4-chlorothien-2-yl | OCH₃ |
| A-57 | 5-chlorothien-2-yl | OCH₃ |
| A-58 | 3-chloro-4-methylthien-2-yl | OCH₃ |
| A-59 | 3-methylthien-2-yl | OCH₃ |
| A-60 | 4-methylthien-2-yl | OCH₃ |
| A-61 | 5-methylthien-2-yl | OCH₃ |
| A-62 | pyridin-2-yl | OCH₃ |
| A-63 | pyridin-3-yl | OCH₃ |
| A-64 | 6-chloropyrid-3-yl | OCH₃ |
| A-65 | 6-bromopyrid-3-yl | OCH₃ |
| A-66 | 6-methylthiopyrid-2-yl | OCH₃ |
| A-67 | 2-fluorophenyl | OCH₃ |
| A-68 | phenyl | OCH₃ |
| A-69 | 6-methylpyrid-2-yl | OCH₃ |
| A-70 | 6-bromopyrid-2-yl | OCH₃ |
| A-71 | 3-fluoropyrid-2-yl | OCH₃ |
| A-72 | pyrid-4-yl | OCH₃ |
| A-73 | 2-thienyl | E—SCH₃ |
| A-74 | 3-thienyl | E—SCH₃ |
| A-75 | 3-bromothien-2-yl | E—SCH₃ |
| A-76 | 4-bromothien-2-yl | E—SCH₃ |
| A-77 | 5-bromothien-2-yl | E—SCH₃ |
| A-78 | 4,5-dibromothien-2-yl | E—SCH₃ |
| A-79 | 3-chlorothien-2-yl | E—SCH₃ |
| A-80 | 4-chlorothien-2-yl | E—SCH₃ |
| A-81 | 5-chlorothien-2-yl | E—SCH₃ |
| A-82 | 3-chloro-4-methylthien-2-yl | E—SCH₃ |
| A-83 | 3-methylthien-2-yl | E—SCH₃ |
| A-84 | 4-methylthien-2-yl | E—SCH₃ |
| A-85 | 5-methylthien-2-yl | E—SCH₃ |
| A-86 | pyridin-2-yl | E—SCH₃ |
| A-87 | pyridin-3-yl | E—SCH₃ |
| A-88 | 6-chloropyrid-3-yl | E—SCH₃ |
| A-89 | 6-bromopyrid-3-yl | E—SCH₃ |
| A-90 | 6-methylthiopyrid-2-yl | E—SCH₃ |
| A-91 | 2-fluorophenyl | E—SCH₃ |
| A-92 | phenyl | E—SCH₃ |
| A-93 | 6-methylpyrid-2-yl | E—SCH₃ |
| A-94 | 6-bromopyrid-2-yl | E—SCH₃ |
| A-95 | 3-fluoropyrid-2-yl | E—SCH₃ |
| A-96 | pyrid-4-yl | E—SCH₃ |
| A-97 | 2-thienyl | Z—SCH₃ |
| A-98 | 3-thienyl | Z—SCH₃ |
| A-99 | 3-bromothien-2-yl | Z—SCH₃ |
| A-100 | 4-bromothien-2-yl | Z—SCH₃ |
| A-101 | 5-bromothien-2-yl | Z—SCH₃ |
| A-102 | 4,5-dibromothien-2-yl | Z—SCH₃ |
| A-103 | 3-chlorothien-2-yl | Z—SCH₃ |
| A-104 | 4-chlorothien-2-yl | Z—SCH₃ |
| A-105 | 5-chlorothien-2-yl | Z—SCH₃ |
| A-106 | 3-chloro-4-methylthien-2-yl | Z—SCH₃ |
| A-107 | 3-methylthien-2-yl | Z—SCH₃ |
| A-108 | 4-methylthien-2-yl | Z—SCH₃ |
| A-109 | 5-methylthien-2-yl | Z—SCH₃ |
| A-110 | pyridin-2-yl | Z—SCH₃ |
| A-111 | pyridin-3-yl | Z—SCH₃ |
| A-112 | 6-chloropyrid-3-yl | Z—SCH₃ |
| A-113 | 6-bromopyrid-3-yl | Z—SCH₃ |
| A-114 | 6-methylthiopyrid-2-yl | Z—SCH₃ |
| A-115 | 2-fluorophenyl | Z—SCH₃ |
| A-116 | phenyl | Z—SCH₃ |
| A-117 | 6-methylpyrid-2-yl | Z—SCH₃ |
| A-118 | 6-bromopyrid-2-yl | Z—SCH₃ |
| A-119 | 3-fluoropyrid-2-yl | Z—SCH₃ |
| A-120 | pyrid-4-yl | Z—SCH₃ |
| A-121 | 2-thienyl | S(O)₂—CH₃ |
| A-122 | 3-thienyl | S(O)₂—CH₃ |
| A-123 | 3-bromothien-2-yl | S(O)₂—CH₃ |
| A-124 | 4-bromothien-2-yl | S(O)₂—CH₃ |
| A-125 | 5-bromothien-2-yl | S(O)₂—CH₃ |
| A-126 | 4,5-dibromothien-2-yl | S(O)₂—CH₃ |
| A-127 | 3-chlorothien-2-yl | S(O)₂—CH₃ |
| A-128 | 4-chlorothien-2-yl | S(O)₂—CH₃ |
| A-129 | 5-chlorothien-2-yl | S(O)₂—CH₃ |
| A-130 | 3-chloro-4-methylthien-2-yl | S(O)₂—CH₃ |
| A-131 | 3-methylthien-2-yl | S(O)₂—CH₃ |
| A-132 | 4-methylthien-2-yl | S(O)₂—CH₃ |
| A-133 | 5-methylthien-2-yl | S(O)₂—CH₃ |
| A-134 | pyridin-2-yl | S(O)₂—CH₃ |
| A-135 | pyridin-3-yl | S(O)₂—CH₃ |
| A-136 | 6-chloropyrid-3-yl | S(O)₂—CH₃ |
| A-137 | 6-bromopyrid-3-yl | S(O)₂—CH₃ |
| A-138 | 6-methylthiopyrid-2-yl | S(O)₂—CH₃ |
| A-139 | 2-fluorophenyl | S(O)₂—CH₃ |
| A-140 | phenyl | S(O)₂—CH₃ |
| A-141 | 6-methylpyrid-2-yl | S(O)₂—CH₃ |
| A-142 | 6-bromopyrid-2-yl | S(O)₂—CH₃ |
| A-143 | 3-fluoropyrid-2-yl | S(O)₂—CH₃ |
| A-144 | pyrid-4-yl | S(O)₂—CH₃ |
| A-145 | 2-thienyl | E—SCH₂CF₃ |
| A-146 | 3-thienyl | E—SCH₂CF₃ |
| A-147 | 3-bromothien-2-yl | E—SCH₂CF₃ |
| A-148 | 4-bromothien-2-yl | E—SCH₂CF₃ |
| A-149 | 5-bromothien-2-yl | E—SCH₂CF₃ |
| A-150 | 4,5-dibromothien-2-yl | E—SCH₂CF₃ |
| A-151 | 3-chlorothien-2-yl | E—SCH₂CF₃ |
| A-152 | 4-chlorothien-2-yl | E—SCH₂CF₃ |
| A-153 | 5-chlorothien-2-yl | E—SCH₂CF₃ |
| A-154 | 3-chloro-4-methylthien-2-yl | E—SCH₂CF₃ |
| A-155 | 3-methylthien-2-yl | E—SCH₂CF₃ |
| A-156 | 4-methylthien-2-yl | E—SCH₂CF₃ |
| A-157 | 5-methylthien-2-yl | E—SCH₂CF₃ |
| A-158 | pyridin-2-yl | E—SCH₂CF₃ |
| A-159 | pyridin-3-yl | E—SCH₂CF₃ |
| A-160 | 6-chloropyrid-3-yl | E—SCH₂CF₃ |
| A-161 | 6-bromopyrid-3-yl | E—SCH₂CF₃ |
| A-162 | 6-methylthiopyrid-2-yl | E—SCH₂CF₃ |
| A-163 | 2-fluorophenyl | E—SCH₂CF₃ |
| A-164 | phenyl | E—SCH₂CF₃ |
| A-165 | 6-methylpyrid-2-yl | E—SCH₂CF₃ |
| A-166 | 6-bromopyrid-2-yl | E—SCH₂CF₃ |
| A-167 | 3-fluoropyrid-2-yl | E—SCH₂CF₃ |
| A-168 | pyrid-4-yl | E—SCH₂CF₃ |
| A-169 | 2-thienyl | Z—SCH₂CF₃ |
| A-170 | 3-thienyl | Z—SCH₂CF₃ |
| A-171 | 3-bromothien-2-yl | Z—SCH₂CF₃ |
| A-172 | 4-bromothien-2-yl | Z—SCH₂CF₃ |
| A-173 | 5-bromothien-2-yl | Z—SCH₂CF₃ |
| A-174 | 4,5-dibromothien-2-yl | Z—SCH₂CF₃ |
| A-175 | 3-chlorothien-2-yl | Z—SCH₂CF₃ |
| A-176 | 4-chlorothien-2-yl | Z—SCH₂CF₃ |
| A-177 | 5-chlorothien-2-yl | Z—SCH₂CF₃ |
| A-178 | 3-chloro-4-methylthien-2-yl | Z—SCH₂CF₃ |
| A-179 | 3-methylthien-2-yl | Z—SCH₂CF₃ |
| A-180 | 4-methylthien-2-yl | Z—SCH₂CF₃ |
| A-181 | 5-methylthien-2-yl | Z—SCH₂CF₃ |
| A-182 | pyridin-2-yl | Z—SCH₂CF₃ |
| A-183 | pyridin-3-yl | Z—SCH₂CF₃ |
| A-184 | 6-chloropyrid-3-yl | Z—SCH₂CF₃ |
| A-185 | 6-bromopyrid-3-yl | Z—SCH₂CF₃ |
| A-186 | 6-methylthiopyrid-2-yl | Z—SCH₂CF₃ |
| A-187 | 2-fluorophenyl | Z—SCH₂CF₃ |
| A-188 | phenyl | Z—SCH₂CF₃ |
| A-189 | 6-methylpyrid-2-yl | Z—SCH₂CF₃ |
| A-190 | 6-bromopyrid-2-yl | Z—SCH₂CF₃ |
| A-191 | 3-fluoropyrid-2-yl | Z—SCH₂CF₃ |
| A-192 | pyrid-4-yl | Z—SCH₂CF₃ |
| A-193 | 2-thienyl | O—C₂H₅ |
| A-194 | 3-thienyl | O—C₂H₅ |
| A—195 | 3-bromothien-2-yl | O—C₂H₅ |
| A-196 | 4-bromothien-2-yl | O—C₂H₅ |
| A-197 | 5-bromothien-2-yl | O—C₂H₅ |
| A-198 | 4,5-dibromothien-2-yl | O—C₂H₅ |
| A-199 | 3-chlorothien-2-yl | O—C₂H₅ |
| A-200 | 4-chlorothien-2-yl | O—C₂H₅ |
| A-201 | 5-chlorothien-2-yl | O—C₂H₅ |
| A-202 | 3-chloro-4-methylthien-2-yl | O—C₂H₅ |
| A-203 | 3-methylthien-2-yl | O—C₂H₅ |
| A-204 | 4-methylthien-2-yl | O—C₂H₅ |
| A-205 | 5-methylthien-2-yl | O—C₂H₅ |
| A-206 | pyridin-2-yl | O—C₂H₅ |
| A-207 | pyridin-3-yl | O—C₂H₅ |
| A-208 | 6-chloropyrid-3-yl | O—C₂H₅ |
| A-209 | 6-bromopyrid-3-yl | O—C₂H₅ |
| A-210 | 6-methylthiopyrid-2-yl | O—C₂H₅ |
| A-211 | 2-fluorophenyl | O—C₂H₅ |
| A-212 | phenyl | O—C₂H₅ |
| A-213 | 6-methylpyrid-2-yl | O—C₂H₅ |
| A-214 | 6-bromopyrid-2-yl | O—C₂H₅ |
| A-215 | 3-fluoropyrid-2-yl | O—C₂H₅ |
| A-216 | pyrid-4-yl | O—C₂H₅ |
| A-217 | 2-thienyl | E—S—C₂H₅ |
| A-218 | 3-thienyl | E—S—C₂H₅ |
| A-219 | 3-bromothien-2-yl | E—S—C₂H₅ |
| A-220 | 4-bromothien-2-yl | E—S—C₂H₅ |
| A-221 | 5-bromothien-2-yl | E—S—C₂H₅ |
| A-222 | 4,5-dibromothien-2-yl | E—S—C₂H₅ |
| A-223 | 3-chlorothien-2-yl | E—S—C₂H₅ |
| A-224 | 4-chlorothien-2-yl | E—S—C₂H₅ |
| A-225 | 5-chlorothien-2-yl | E—S—C₂H₅ |
| A-226 | 3-chloro-4-methylthien-2-yl | E—S—C₂H₅ |
| A-227 | 3-methylthien-2-yl | E—S—C₂H₅ |
| A-228 | 4-methylthien-2-yl | E—S—C₂H₅ |
| A-229 | 5-methylthien-2-yl | E—S—C₂H₅ |
| A-230 | pyridin-2-yl | E—S—C₂H₅ |
| A-231 | pyridin-3-yl | E—S—C₂H₅ |
| A-232 | 6-chloropyrid-3-yl | E—S—C₂H₅ |
| A-233 | 6-bromopyrid-3-yl | E—S—C₂H₅ |
| A-234 | 6-methylthiopyrid-2-yl | E—S—C₂H₅ |
| A-235 | 2-fluorophenyl | E—S—C₂H₅ |
| A-236 | phenyl | E—S—C₂H₅ |
| A-237 | 6-methylpyrid-2-yl | E—S—C₂H₅ |
| A-238 | 6-bromopyrid-2-yl | E—S—C₂H₅ |
| A-239 | 3-fluoropyrid-2-yl | E—S—C₂H₅ |
| A-240 | pyrid-4-yl | E—S—C₂H₅ |
| A-241 | 2-thienyl | Z—S—C₂H₅ |
| A-242 | 3-thienyl | Z—S—C₂H₅ |
| A-243 | 3-bromothien-2-yl | Z—S—C₂H₅ |
| A-244 | 4-bromothien-2-yl | Z—S—C₂H₅ |
| A-245 | 5-bromothien-2-yl | Z—S—C₂H₅ |
| A-246 | 4,5-dibromothien-2-yl | Z—S—C₂H₅ |
| A-247 | 3-chlorothien-2-yl | Z—S—C₂H₅ |
| A-248 | 4-chlorothien-2-yl | Z—S—C₂H₅ |
| A-249 | 5-chlorothien-2-yl | Z—S—C₂H₅ |
| A-250 | 3-chloro-4-methylthien-2-yl | Z—S—C₂H₅ |
| A-251 | 3-methylthien-2-yl | Z—S—C₂H₅ |
| A-252 | 4-methylthien-2-yl | Z—S—C₂H₅ |
| A-253 | 5-methylthien-2-yl | Z—S—C₂H₅ |
| A-254 | pyridin-2-yl | Z—S—C₂H₅ |
| A-255 | pyridin-3-yl | Z—S—C₂H₅ |
| A-256 | 6-chloropyrid-3-yl | Z—S—C₂H₅ |
| A-257 | 6-bromopyrid-3-yl | Z—S—C₂H₅ |
| A-258 | 6-methylthiopyrid-2-yl | Z—S—C₂H₅ |
| A-259 | 2-fluorophenyl | Z—S—C₂H₅ |
| A-260 | phenyl | Z—S—C₂H₅ |
| A-261 | 6-methylpyrid-2-yl | Z—S—C₂H₅ |
| A-262 | 6-bromopyrid-2-yl | Z—S—C₂H₅ |
| A-263 | 3-fluoropyrid-2-yl | Z—S—C₂H₅ |
| A-264 | pyrid-4-yl | Z—S—C₂H₅ |
| A-265 | 2-thienyl | E—S—CH(CH₃)₂ |
| A-266 | 3-thienyl | E—S—CH(CH₃)₂ |
| A-267 | 3-bromothien-2-yl | E—S—CH(CH₃)₂ |
| A-268 | 4-bromothien-2-yl | E—S—CH(CH₃)₂ |
| A-269 | 5-bromothien-2-yl | E—S—CH(CH₃)₂ |
| A-270 | 4,5-dibromothien-2-yl | E—S—CH(CH₃)₂ |
| A-271 | 3-chlorothien-2-yl | E—S—CH(CH₃)₂ |
| A-272 | 4-chlorothien-2-yl | E—S—CH(CH₃)₂ |
| A-273 | 5-chlorothien-2-yl | E—S—CH(CH₃)₂ |
| A-274 | 3-chloro-4-methylthien-2-yl | E—S—CH(CH₃)₂ |
| A-275 | 3-methylthien-2-yl | E—S—CH(CH₃)₂ |
| A-276 | 4-methylthien-2-yl | E—S—CH(CH₃)₂ |
| A-277 | 5-methylthien-2-yl | E—S—CH(CH₃)₂ |
| A-278 | pyridin-2-yl | E—S—CH(CH₃)₂ |
| A-279 | pyridin-3-yl | E—S—CH(CH₃)₂ |
| A-280 | 6-chloropyrid-3-yl | E—S—CH(CH₃)₂ |
| A-281 | 6-bromopyrid-3-yl | E—S—CH(CH₃)₂ |
| A-282 | 6-methylthiopyrid-2-yl | E—S—CH(CH₃)₂ |
| A-283 | 2-fluorophenyl | E—S—CH(CH₃)₂ |
| A-284 | phenyl | E—S—CH(CH₃)₂ |
| A-285 | 6-methylpyrid-2-yl | E—S—CH(CH₃)₂ |
| A-286 | 6-bromopyrid-2-yl | E—S—CH(CH₃)₂ |
| A-287 | 3-fluoropyrid-2-yl | E—S—CH(CH₃)₂ |
| A-288 | pyrid-4-yl | E—S—CH(CH₃)₂ |
| A-289 | 2-thienyl | Z—S—CH(CH₃)₂ |
| A-290 | 3-thienyl | Z—S—CH(CH₃)₂ |
| A-291 | 3-bromothien-2-yl | Z—S—CH(CH₃)₂ |
| A-292 | 4-bromothien-2-yl | Z—S—CH(CH₃)₂ |
| A-293 | 5-bromothien-2-yl | Z—S—CH(CH₃)₂ |
| A-294 | 4,5-dibromothien-2-yl | Z—S—CH(CH₃)₂ |
| A-295 | 3-chlorothien-2-yl | Z—S—CH(CH₃)₂ |
| A-296 | 4-chlorothien-2-yl | Z—S—CH(CH₃)₂ |
| A-297 | 5-chlorothien-2-yl | Z—S—CH(CH₃)₂ |
| A-298 | 3-chloro-4-methylthien-2-yl | Z—S—CH(CH₃)₂ |
| A-299 | 3-methylthien-2-yl | Z—S—CH(CH₃)₂ |
| A-300 | 4-methylthien-2-yl | Z—S—CH(CH₃)₂ |
| A-301 | 5-methylthien-2-yl | Z—S—CH(CH₃)₂ |
| A-302 | pyridin-2-yl | Z—S—CH(CH₃)₂ |
| A-303 | pyridin-3-yl | Z—S—CH(CH₃)₂ |
| A-304 | 6-chloropyrid-3-yl | Z—S—CH(CH₃)₂ |
| A-305 | 6-bromopyrid-3-yl | Z—S—CH(CH₃)₂ |
| A-306 | 6-methylthiopyrid-2-yl | Z—S—CH(CH₃)₂ |
| A-307 | 2-fluorophenyl | Z—S—CH(CH₃)₂ |
| A-308 | phenyl | Z—S—CH(CH₃)₂ |
| A-309 | 6-methylpyrid-2-yl | Z—S—CH(CH₃)₂ |
| A-310 | 6-bromopyrid-2-yl | Z—S—CH(CH₃)₂ |
| A-311 | 3-fluoropyrid-2-yl | Z—S—CH(CH₃)₂ |
| A-312 | pyrid-4-yl | Z—S—CH(CH₃)₂ |
| A-313 | 2-thienyl | E—S—CH₂CH₂CH₃ |
| A-314 | 3-thienyl | E—S—CH₂CH₂CH₃ |
| A-315 | 3-bromothien-2-yl | E—S—CH₂CH₂CH₃ |
| A-316 | 4-bromothien-2-yl | E—S—CH₂CH₂CH₃ |
| A-317 | 5-bromothien-2-yl | E—S—CH₂CH₂CH₃ |
| A-318 | 4,5-dibromothien-2-yl | E—S—CH₂CH₂CH₃ |
| A-319 | 3-chlorothien-2-yl | E—S—CH₂CH₂CH₃ |
| A-320 | 4-chlorothien-2-yl | E—S—CH₂CH₂CH₃ |
| A-321 | 5-chlorothien-2-yl | E—S—CH₂CH₂CH₃ |
| A-322 | 3-chloro-4-methylthien-2-yl | E—S—CH₂CH₂CH₃ |
| A-323 | 3-methylthien-2-yl | E—S—CH₂CH₂CH₃ |
| A-324 | 4-methylthien-2-yl | E—S—CH₂CH₂CH₃ |
| A-325 | 5-methylthien-2-yl | E—S—CH₂CH₂CH₃ |
| A-326 | pyridin-2-yl | E—S—CH₂CH₂CH₃ |
| A-327 | pyridin-3-yl | E—S—CH₂CH₂CH₃ |
| A-328 | 6-chloropyrid-3-yl | E—S—CH₂CH₂CH₃ |
| A-329 | 6-bromopyrid-3-yl | E—S—CH₂CH₂CH₃ |
| A-330 | 6-methylthiopyrid-2-yl | E—S—CH₂CH₂CH₃ |
| A-331 | 2-fluorophenyl | E—S—CH₂CH₂CH₃ |
| A-332 | phenyl | E—S—CH₂CH₂CH₃ |
| A-333 | 6-methylpyrid-2-yl | E—S—CH₂CH₂CH₃ |
| A-334 | 6-bromopyrid-2-yl | E—S—CH₂CH₂CH₃ |
| A-335 | 3-fluoropyrid-2-yl | E—S—CH₂CH₂CH₃ |
| A-336 | pyrid-4-yl | E—S—CH₂CH₂CH₃ |
| A-337 | 2-thienyl | Z—S—CH₂CH₂CH₃ |
| A-338 | 3-thienyl | Z—S—CH₂CH₂CH₃ |
| A-339 | 3-bromothien-2-yl | Z—S—CH₂CH₂CH₃ |
| A-340 | 4-bromothien-2-yl | Z—S—CH₂CH₂CH₃ |
| A-341 | 5-bromothien-2-yl | Z—S—CH₂CH₂CH₃ |
| A-342 | 4,5-dibromothien-2-yl | Z—S—CH₂CH₂CH₃ |
| A-343 | 3-chlorothien-2-yl | Z—S—CH₂CH₂CH₃ |
| A-344 | 4-chlorothien-2-yl | Z—S—CH₂CH₂CH₃ |
| A-345 | 5-chlorothien-2-yl | Z—S—CH₂CH₂CH₃ |
| A-346 | 3-chloro-4-methylthien-2-yl | Z—S—CH₂CH₂CH₃ |
| A-347 | 3-methylthien-2-yl | Z—S—CH₂CH₂CH₃ |
| A-348 | 4-methylthien-2-yl | Z—S—CH₂CH₂CH₃ |
| A-349 | 5-methylthien-2-yl | Z—S—CH₂CH₂CH₃ |
| A-350 | pyridin-2-yl | Z—S—CH₂CH₂CH₃ |
| A-351 | pyridin-3-yl | Z—S—CH₂CH₂CH₃ |
| A-352 | 6-chloropyrid-3-yl | Z—S—CH₂CH₂CH₃ |
| A-353 | 6-bromopyrid-3-yl | Z—S—CH₂CH₂CH₃ |
| A-354 | 6-methylthiopyrid-2-yl | Z—S—CH₂CH₂CH₃ |
| A-355 | 2-fluorophenyl | Z—S—CH₂CH₂CH₃ |
| A-356 | phenyl | Z—S—CH₂CH₂CH₃ |
| A-357 | 6-methylpyrid-2-yl | Z—S—CH₂CH₂CH₃ |
| A-358 | 6-bromopyrid-2-yl | Z—S—CH₂CH₂CH₃ |
| A-359 | 3-fluoropyrid-2-yl | Z—S—CH₂CH₂CH₃ |
| A-360 | pyrid-4-yl | Z—S—CH₂CH₂CH₃ |
| A-361 | 2-thienyl | CH₂CH₃ |
| A-362 | 3-thienyl | CH₂CH₃ |
| A-363 | 3-bromothien-2-yl | CH₂CH₃ |
| A-364 | 4-bromothien-2-yl | CH₂CH₃ |
| A-365 | 5-bromothien-2-yl | CH₂CH₃ |
| A-366 | 4,5-dibromothien-2-yl | CH₂CH₃ |
| A-367 | 3-chlorothien-2-yl | CH₂CH₃ |
| A-368 | 4-chlorothien-2-yl | CH₂CH₃ |
| A-369 | 5-chlorothien-2-yl | CH₂CH₃ |
| A-370 | 3-chloro-4-methylthien-2-yl | CH₂CH₃ |
| A-371 | 3-methylthien-2-yl | CH₂CH₃ |
| A-372 | 4-methylthien-2-yl | CH₂CH₃ |
| A-373 | 5-methylthien-2-yl | CH₂CH₃ |
| A-374 | pyridin-2-yl | CH₂CH₃ |
| A-375 | pyridin-3-yl | CH₂CH₃ |
| A-376 | 6-chloropyrid-3-yl | CH₂CH₃ |
| A-377 | 6-bromopyrid-3-yl | CH₂CH₃ |
| A-378 | 6-methylthiopyrid-2-yl | CH₂CH₃ |
| A-379 | 6-methylpyrid-2-yl | CH₂CH₃ |
| A-380 | 6-bromopyrid-2-yl | CH₂CH₃ |
| A-381 | 3-fluoropyrid-2-yl | CH₂CH₃ |
| A-382 | pyrid-4-yl | CH₂CH₃ |
| A-383 | 2-fluorophenyl | CH₂CH₃ |
| A-384 | phenyl | CH₂CH₃ |

In table A and in the working examples the denominators E and Z refer to the configuration of the C(X)═N double bond in formula I, i.e. to the relative spatial arrangement of the moieties A-(CH₂)ₙ and —N═C(R¹)— with, respect to the double bond C(X)═N.

Another very preferred embodiment of the invention relates to compounds of the general formula Ib: 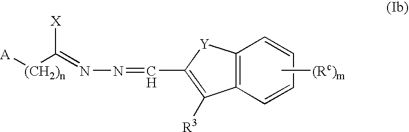
wherein m is 0, 1, 2 or 3, Y is O or S and wherein A, n, X, R³ and R^{c} are as defined above. Amongst the compounds Ib those are preferred, wherein n is 0 and wherein A, X, R³ and R^{c} have the meanings given as preferred.

Examples of compounds Ib are given in the following tables 91 to 102:

Table 91:

Compounds of the formula Ib, wherein n is 0, Y is O and R³ is H and m is 0 (i.e. (R^{c})ₘ is absent) and wherein X and A are given in table A;

Table 92:

Compounds of the formula Ib, wherein n is 0, Y is O and R³ is H and (R^{c})ₘ is 5-fluoro and wherein X and A are given in table A;

Table 93:

Compounds of the formula Ib, wherein n is 0, Y is O and R³ is H and (R^{c})ₘ is 5-chloro and wherein X and A are given in table A;

Table 94:

Compounds of the formula Ib, wherein n is 0, Y is O and R³ is CH₃ and m is 0 (i.e. (R^{c})ₘ is absent) and wherein X and A are given in table A;

Table 95:

Compounds of the formula Ib, wherein n is 0, Y is O and R³ is CH₃ and (R^{c})ₘ is 5-fluoro and wherein X and A are given in table A;

Table 96:

Compounds of the formula Ib, wherein n is 0, Y is O and R³ is CH₃ and (R^{c})ₘ is 5-chloro and wherein X and A are given in table A;

Table 97:

Compounds of the formula Ib, wherein n is 0, Y is S and R³ is H and m is 0 (i.e. (R^{c})ₘ is absent) and wherein X and A are given in table A;

Table 98:

Compounds of the formula Ib, wherein n is 0, Y is S and R³ is H and (R^{c})ₘ is 5-fluoro and wherein X and A are given in table A;

Table 99:

Compounds of the formula Ib, wherein n is 0, Y is S and R³ is H and (R^{c})ₘ is 5-chloro and wherein X and A are given in table A;

Table 100:

Compounds of the formula Ib, wherein n is 0, Y is S and R³ is CH₃ and m is 0 (i.e. (R^{c})ₘ is absent) and wherein X and A are given in table A;

Table 101:

Compounds of the formula Ib, wherein n is 0, Y is S and R³ is CH₃ and (R^{c})ₘ is 5-fluoro and wherein X and A are given in table A;

Table 102:

Compounds of the formula Ib, wherein n is 0, Y is S and R³ is CH₃ and (R^{c})ₘ is 5-chloro and wherein X and A are given in table A;

A further preferred embodiment of the invention relates to compounds of the general formula Ic: 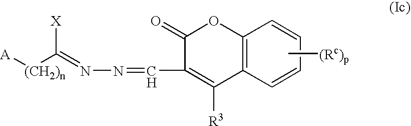
wherein p is 0, 1, 2 or 3, and wherein A, n, X, R³ and R^{c} are as defined above. Amongst the compounds Ic those are preferred, wherein n is 0 and wherein A, X, R³ and R^{c} have the meanings given as preferred.

Examples of compounds Ic are given in table 103

Table 103:

Compounds of the formula Ic, wherein n is 0 and R³ is H and p is 0 (i.e. (R^{c})ₚ is absent) and wherein X and A are given in table A;

A further preferred embodiment of the invention relates to compounds of the general formula Id: 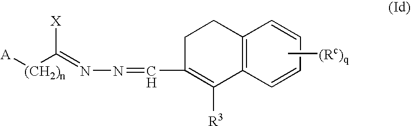
wherein q is 0, 1, 2 or 3, and wherein A, n, X, R³ and R^{c} are as defined above. Amongst the compounds Id those are preferred, wherein n is 0 and wherein A, X, R³ and R^{c} have the meanings given as preferred.

Examples of compounds Id are given in table 104.

Table 104:

Compounds of the formula Id, wherein n is 0 and R³ is H and q is 0 (i.e. (R^{c})_{q} is absent) and wherein X and A are given in table A;

The compounds of the formula I may be readily synthesized using techniques generally known by synthetic organic chemists.

Compounds of the formula I, wherein X is halogen (Hal) can be prepared from acylhydrazone compounds of the general formula II according to scheme 1:

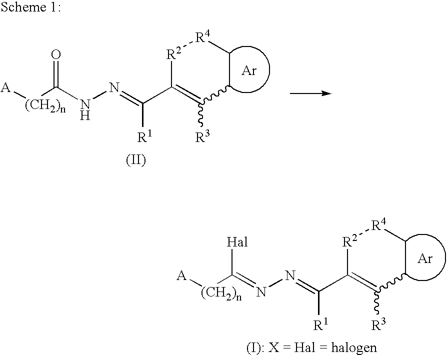

According to scheme 1, compounds of formula II are converted to compounds of the formula I, wherein X is halogen, in particular chlorine. The reaction depicted in scheme 1 can be performed by analogy to known methods such as described in J. Fluorine Chem. 1983, 23, 293-299 or J. Org. Chem. 1993, 58, 32-35.

The compounds of the formula I with X=halogen can be converted into other compounds of formula I by replacement of the halogen atom by nucleophiles under standard conditions (see scheme 2):

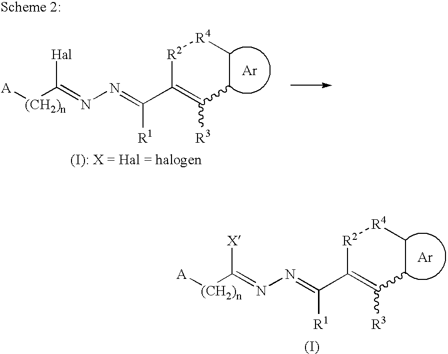

In scheme 2 the variables A, n, Hal, Ar, R¹, R², R³ and R⁴ are as defined above. X′ has one of the meanings given for X except for halogen. Suitable reactions for replacing halogen by other nucleophiles are e.g. described in Heterocycles, 1993, 36(7), 1471-1476, Tetrahedron Lett. 1985, 26(29), 3463-3466 or Tetrahedron Lett. 2003, 44(47), 8577-8580. The reaction conditions described therein can be applied to the reaction depicted in scheme 2 by analogy.

If individual compounds I are not obtainable by the route described above, they can be prepared by derivatization of other compounds I or by customary modifications of the synthesis routes described. The preparation of the compounds of formula I may lead to them being obtained as isomer mixtures (stereoisomers, enantiomers). If desired, these can be resolved by the methods customary for this purpose, such as crystallization or chromatography, also on optically active adsorbate, to give the pure isomers.

Acyl hydrazones of the formula II are known in the art, e.g. from PCT/EP 2004/005681, or they can be obtained applying synthesis methods described for example in WO 87/06133 by analogy. For Instance, suitable acyl hydrazides can be reacted with aldehydes, esters or ketones according to scheme 3 to form acyl hydrazones of the formula II.

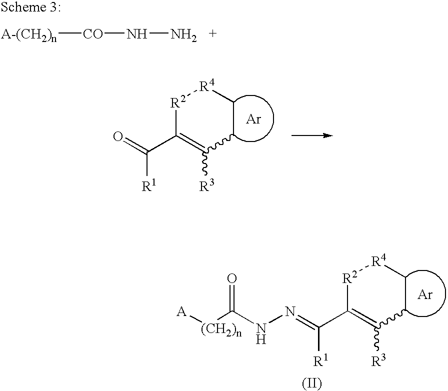

The compounds of formula I are effective through contact (via soil, glass, wall, bed net, carpet, plant parts or animal parts), and/or Ingestion (bait, or plant part).

The compounds of the formula I are in particular suitable for efficiently controlling nematodes and insects. In particular, they are suitable for controlling the following pests:

Insects:

from the order of the lepidopterans (Lepidoptera), for example Agrotis upsilon, Agrotis segetum, Alabama argillacea, Anticarsia gemmatalis, Argyresthia conjugella, Autographa gamma, Bupalus piniarius, Cacoecia murinana, Capua reticulana, Chematobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranean, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera scftella, Lithocolletis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthonrmaea operculella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis pilleriana, Spodoptera frugiperda, Spodoptera littoralis, Spodoptera litura, Thaumatopoea pityocampa, Tortrix viridana, Trichoplusia ni and Zeiraphera canadensis,

beetles (Coleoptera), for example Agrilus sinuatus, Agriotes lineatus, Agriotes obscurus, Amphimallus solstftialis, Anisandrus dispar, Anthonomus grandis, Anthonomus pomorum, Atomaria linearis, Blastophagus piniperda, BEitophaga undata, Bruchus rufimanus, Bruchus pisorum, Bruchus lentis, Byctiscus betulae, Cassida nebulosa, Cerotoma trifurcata, Ceuthorrhynchus assimilis, Ceuthorrhynchus napi, Chaetocnema tibialis, Conoderus vespertinus, Crioceris asparagi, Diabrotica longicomis, Diabrotica 12-punctata, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Ortiorrhynchus sulcatus, Otiorrhynchus ovatus, Phaedon cochleariae, Phyllotreta chrysocephala, Phyllophaga sp., Phyllopertha horticola, Phyllotreta nemorum, Phyllotreta striolata, Popillia japonica, Sitona lineatus and Sitophilus granaria,

dipterans (Diptera), for example Aedes aegypti, Aedes vexans, Anastrepha ludens, Anopheles maculipennis, Ceratitis capitata, Chrysomya bezziana, Chrysomya hominivorax, Chrysomya macellaria, Contarinia sorghicola, Cordylobia anthropophaga, Culexpipiens, Dacus cucurbitae, Dacus oleae, Dasineura brassicae, Fannia canicularis, Gasterophilus intestinalis, Glossina morsitans, Haematobia irritans, Haplodiplosis equestris, Hylemyia platura, Hypoderma lineata, Liriomyza sativae, Liriomyza trifolii, Lucilia caprina, Lucilia cuprina, Lucilia sericata, Lycoria pectoralis, Mayetiola destructor, Musca domestica, Muscina stabulans, Oestrus ovis, Oscinella frit, Pegomya hysocyami, Phorbia antiqua, Phorbia brassicae, Phorbia coarctata, Rhagoletis cerasi, Rhagotetis pomonella, Tabanus bovinus, Tipula oleracea and Tipula paludosa,

thrips (Thysanoptera), e.g. Dichromothrips spp., Frankliniella fusca, Frankliniella occidentalis, Frankilniella tritici, Scirtothrips citri, Thrips oryzae, Thrips palmi and Thrips tabaci,

hymenopterans (Hymenoptera), e.g. Athalia rosae, Atta cephalotes, Atta sexdens, Atta texana, Hoplocampa minuta, Hoplocampa testudinea, Monomorium pharaonis, Solenopsis geminata and Solenopsis invicta,

heteropterans (Heteroptera), e.g. Acrostemum hilare, Blissus leucopterus, Cyrtopeltis notatus, Dysdercus cingulatus, Dysdercus internedius, Eurygaster integriceps, Euschistus impictiventris, Leptoglossus phyllopus, Lygus lineolaris, Lygus pratensis, Nezara viridula, Piesma quadrata, Solubea insularis and Thyanta perditor,

homopterans (Homoptera), e.g. Acyrthosiphon onobrychis, Adelges laricis, Aphidula nasturtil, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Bemisa tabaci, Bemisa argentifolii, Brachycaudus cardui, Brachycaudus helichrysi, Brachycaudus persicae, Brachycaudus prunicola, Brevicoryne brassicae, Capitophorus homi, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzodes persicae, Myzus ascalonicus, Myzus cerasi, Myzus varians, Nasonovia ribis-nigri, Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantiand, and Vfteus vitifolii,

termites (Isoptera), e.g. Calotermes flavicollis, Leucoternes flavipes, Reticulitermes lucifugus und Termes natalensis, and

orthopterans (Orthoptera), e.g. Acheta domestica, Blatta orientalis, Blattella germanica, Forficula auricularia, Gryllotalpa gryllotalpa, Locusta migratoria, Melanoplus bivittatus, Melanoplus femur-rubrum, Melanoplus mexicanus, Melanoplus sanguinipes, Melanoplus spretus, Nomadacris septemfasciata, Periplaneta americana, Schistocerca americana, Schistocerca peregrina, Stauronotus maroccanus and Tachycines asynamorus;

Nematodes:

plant parasitic nematodes such as root knot nematodes, Meloidogyne hapla, Meloidogyne incognita, Meloidogyne javanica, and other Meloidogyne species; cyst-forming nematodes, Globodera rostochiensis and other Globodera species; Heterodera avenae, Heterodera glycines, Heterodera schachti, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema ispecies, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmannietla species; Lance nematodes, Hoploaimus species; false rootknot nematodes, Nacobbus species; Needle nematodes, Longidorus elongatus and other Longidorus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus-nematodes, Tylenchulus species; Dagger nematodes, Xiphinema species; and other plant parasitic nematode species.

The compounds of the formula I and their salts are also useful for controlling arachnids (Arachnoidea), such as acarians (Acarina), e.g. of the families Argasidae, Ixodidae and Sarcoptidae, such as Amblyomma americanum, Amblyomma variegatum, Argas persicus, Boophilus annulatus, Boophilus decoloratus, Boophilus microplus, Dermacentor silvarum, Hyalomma truncatum, Ixodes ricinus, Ixodes rubicundus, Ornithodorus moubata, Otobius megnini, Denmanyssus gallinae, Psoroptes ovis, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni; Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus uricae, Panonychus ulmi, Panonychus citri, and oligonychus pratensis.

For use in a method according to the present invention, the compounds I can be converted into the customary formulations, e.g. solutions, emulsions, suspensions, dusts, powders, pastes and granules. The use form depends on the particular purpose; it is intended to ensure in each case a fine and uniform distribution of the compound according to the invention.

The formulations are prepared in a known manner, e.g. by extending the active ingredient with solvents and/or carriers, if desired using emulsifiers and dispersants. Solvents/auxiliaries, which are suitable, are essentially:
water, aromatic solvents (for example Solvesso products, xylene), paraffins (for example mineral fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used.
carriers such as ground natural minerals (e.g. kaolins, clays, talc, chalk) and ground synthetic minerals (e.g. highly disperse silica, silicates); emulsifiers such as nonionic and anionic emulsifiers (e.g. polyoxyethylene fatty alcohol ethers, alkylsulfonates and arylsulfonates) and dispersants such as lignin-sulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignin-sulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dusts can be prepared by mixing or concomitantly grinding the active substances with a solid carrier.

Granules, for example coated granules, impregnated granules and homogeneous granules, can be prepared by binding the active ingredients to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active ingredient. The active ingredients are employed in a purity of from 90% to 100%, preferably 95% to 100% (according to NMR spectrum).

The following are examples of formulations: 1. Products for dilution with water

A Soluble Concentrates (SL)

10 parts by weight of a compound according to the invention are dissolved in water or in a water-soluble solvent. As an alternative, wetters or other auxiliaries are added. The active ingredient dissolves upon dilution with water.

B Dispersible Concentrates (DC)

20 parts by weight of a compound according to the invention are dissolved in cyclohexanone with addition of a dispersant, for example polyvinylpyrrolidone. Dilution with water gives a dispersion.

C Emulsifiable Concentrates (EC)

15 parts by weight of a compound according to the invention are dissolved in xylene with addition of calcium dodecylbenzenesulfonate and castor oil ethoxylate (in each case 5% strength). Dilution with water gives an emulsion.

D Emulsions (EW, EO)

40 parts by weight of a compound according to the invention are dissolved in xylene with addition of calcium dodecylbenzenesulfonate and castor oil ethoxylate (in each case 5% strength). This mixture is introduced into water by means of an emulsifier (Ultraturrax) and made into a homogeneous emulsion. Dilution with water gives an emulsion.

E Suspensions (SC, OD)

In an agitated ball mill, 20 parts by weight of a compound according to the invention are milled with addition of dispersant, wetters and water or an organic solvent to give a fine active ingredient suspension. Dilution with water gives a stable suspension of the active ingredient.

F Water-Dispersible Granules and Water-Soluble Granules (WG, SG)

50 parts by weight of a compound according to the invention are ground finely with addition of dispersants and wetters and made into water-dispersible or water-soluble granules by means of technical appliances (for example extrusion, spray tower, fluidized bed). Dilution with water gives a stable dispersion or solution of the active ingredient.

G Water-Dispersible Powders and Water-Soluble Powders (WP, SP)

75 parts by weight of a compound according to the invention are ground in a rotor-stator mill with addition of dispersant, wetters and silica gel. Dilution with water gives a stable dispersion or solution with the active ingredient.

2. Products to be Applied Undiluted

H Dustable Powders (DP)

5 parts by weight of a compound according to the invention are ground finely and mixed intimately with 95% of finely divided kaolin. This gives a dustable product.

I Granules (GR, FG, GG, MG)

0.5 parts by weight of a compound according to the invention is ground finely and associated with 95.5% carriers. Current methods are extrusion, spray drying or the fluidized bed. This gives granules to be applied undiluted.

J ULV Solutions (UL)

10. parts by weight of a compound according to the invention are dissolved in an organic solvent, for example xylene. This gives a product to be applied undiluted.

The active ingredients can be used as such, in the form of their formulations or the use forms prepared therefrom, e.g. in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; it is intended to ensure in each case the finest possible distribution of the active ingredients according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active ingredient concentrations in the ready-to-use products can be varied within relatively wide ranges. In general, they are from 0.0001 to 10%, preferably from 0.01 to 1%.

The active ingredients may also be used successfully in the ultra-low-volume process (ULV), it being possible to apply formulations comprising over 95% by weight of active ingredient, or even to apply the active ingredient without additives.

Compositions of this invention may also contain other active ingredients, for example other pesticides, insecticides, herbicides, fertilizers such as ammonium nitrate, urea, potash, and superphosphate, phytotoxicants and plant growth regulators, safeners and nematicides. These additional ingredients may be used sequentially or in combination with the above-described compositions, if appropriate also added only immediately prior to use (tank mix). For example, the plant(s) may be sprayed with a composition of this invention either before or after being treated with other active ingredients. These agents usually are admixed with the agents according to the invention in a weight ratio of 1:100 to 100:1.

The following list of pesticides together with which the compounds according to the invention can be used, is intended to illustrate the possible combinations, but not to impose any limitation:

Organophosphates: Acephate, Azinphos-methyl, Chlorpyrifos, Chlorfenvinphos, Diazinon, Dichlorvos, Dicrotophos, Dimethoate, Disulfoton, Ethion, Fenitrothion, Fenthion, Isoxathion, Malathion, Methamidophos, Methidathion, Methyl-Parathion, Mevinphos, Monocrotophos, Oxydemeton-methyl, Paraoxon, Parathion, Phenthoate, Phosalone, Phosmet, Phosphamidon, Phorate, Phoxim, Pirimiphos-methyl, Protenofos, Prothiofos, Sulprophos, Tetrachlorvinphos, Terbufos, Triazophos, Trichlorfon;

Carbamates: Alanycarb, Bendiocarb, Benfuracarb, Carbaryl, Carbofuran, Carbosulfan, Fenoxycarb, Furathiocarb, Indoxacarb, Methiocarb, Methomyl, Oxamyl, Pirimicarb, Propoxur, Thiodicarb, Triazamate;

Pyrethroids: Bifenthrin, Cyfluthrin, Cypermethrin, alpha-Cypermethrin, Deltamethrin, Esfenvalerate, Ethofenprox, Fenpropathrin, Fenvalerate, Cyhalothrin, Lambda-Cyhalothrin, Permethrin, Silafluofen, Tau-Fluvalinate, Tefluthrin, Tralomethrin, Zeta-Cypermethrin;

Arthropod growth regulators: a) chitin synthesis inhibitors: benzoylureas: Chlorfluazuron, Diflubenzuron, Flucycloxuron, Flufenoxuron, Hexaflumuron, Lufenuron, Novaluron, Teflubenzuron, Triflumuron; Buprofezin, Diofenolan, Hexythiazox, Etoxazole, Clofentazine; b) ecdysone antagonists: Halofenozide, Methoxyfenozide, Tebufenozide; c) juvenoids: Pyriproxyfen, Methoprene, Fenoxycarb; d) lipid biosynthesis inhibitors: Spirodiclofen;
Various: Abamectin, Acequinocyl, Acetamiprid, Amitraz, Azadirachtin, Bifenazate, Cartap, Chlorfenapyr, Chlordimeform, Cyromazine, Diafenthiuron, Dinetofuran, Diofenolan, Emamectin, Endosulfan, Ethiprole, Fenazaquin, Fipronil, Formetanate, Formetanate hydrochloride, Hydramethylnon, Imidacloprid, Indoxacarb, Metaflumizon (=4-{(2Z)-2-({[4-(trifluoro-methoxy)anilino]carbonyl}hydrazono)-2-[3-(trifluoromethyl)-phenyl]ethyl}benzo-nitrile), Nitenpyram, Pyridaben, Pymetrozine, Spinosad, Sulfur, Tebufenpyrad, Thiamethoxam, Thiacloprid, Thiocyclam, Spiromesifen, Spirodiclofen, Pyridalyl and the pesticide of the following formula as described in WO 98/05638:

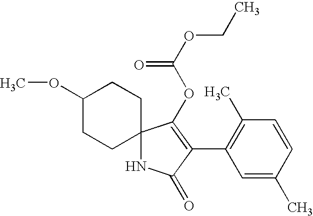

Fungicides are those selected from the group consisting of
acylalanines such as benalaxyl, metalaxyl, ofurace, oxadixyl,
amine derivatives such as aldimorph, dodine, dodemorph, fenpropimorph, fenpropidin, guazatine, iminoctadine, spiroxamin, tridemorph
anilinopyrimidines such as pyrimethanil, mepanipyrim or cyrodinyl,
antibiotics such as cycloheximid, griseofulvin, kasugamycin, natamycin, polyoxin or streptomycin,
azoles such as bitertanol, bromoconazole, cyproconazole, difenoconazole, dinitroconazole, epoxiconazole, fenbuconazole, fluquiconazole, flusilazole, hexaconazole, imazalil, metconazole, myclobutanil, penconazole, propiconazole, prochloraz, prothioconazole, tebuconazole, triadimefon, triadimenol, triflumizol, triticonazole, flutriafol,
dicarboximides such as iprodion, myclozolin, procymidon, vinclozolin,
dithiocarbamates such as ferbam, nabam, maneb, mancozeb, metam, metiram, propineb, polycarbamate, thiram, ziram, zineb,
heterocyclic compounds such as anilazine, benomyl, boscalid, carbendazim, carboxin, oxycarboxin, cyazofamid, dazomet, dithianon, famoxadon, fenamidon, fenarimol, fuberidazole, flutolanil, furametpyr, Isoprothiolane, mepronil, nuarimol, probenazole, proquinazid, pyrifenox, pyroquilon, quinoxyfen, suithiofam, thiabendazole, thifluzamid, thiophanate-methyl, tiadinil, tricyclazole, triforine,
copper fungicides such as Bordeaux mixture, copper acetate, copper oxychloride, basic copper sulfate,
nitrophenyl derivatives such as binapacryl, dinocap, dinobuton, nitrophthalisopropyl
phenylpyrroles such as fenpiclonil or fludioxonil,
sulfur
other fungicides such as acibenzolar-S-methyl, benthiavalicarb, carpropamid, chlorothalonil, cyflufenamid, cymoxanil, diclomezin, diclocymet, diethofencarb, edifenphos, ethaboxam, fenhexamid, fentin-acetate, fenoxanil, ferimzone, fluazinam, fosetyl, fosetyl-aluminum, iprovalicarb, hexachlorobenzene, metrafenon, pencycuron, propamocarb, phthalide, toloclofos-methyl, quintozene, zoxamid
strobilurins such as azoxystrobin, dimoxystrobin, fluoxastrobin, kresoxim-methyl, metominostrobin, orysastrobin, picoxystrobin or trifloxystrobin,
sulfenic acid derivatives such as captafol, captan, dichlofluanid, folpet, tolylfluanid
cinnemamides and analogs such as dimethomorph, flumetover or flumorph.

The aforementioned compositions are particularly useful for protecting plants against infestation of said pests or to combat these pests in infested plants. However, the compounds of formula I are also suitable for the treatment of seeds.

Compositions for seed treatments include for example flowable concentrates FS, solutions LS, powders for dry treatment DS, water dispersible powders for slurry treatment WS, water soluble powders SS and emulsion ES. Application to the seeds is carried out before sowing, either directly on the seeds or after having pregerminated the latter.

Preferred FS formulations of compounds of formula I for seed treatment usually comprise from 0.5 to 80% of the active ingredient, from 0.05 to 5% of a wetter, from 0.5 to 15% of a dispersing agent, from 0, 1 to 5% of a thickener, from 5 to 20% of an anti-freeze agent, from 0, 1 to 2% of an anti-foam agent, from 1 to 20% of a pigment and/or a dye, from 0 to 15% of a sticker/adhesion agent, from 0 to 75% of a filler/vehicle, and from 0.01 to 1% of a preservative.

Suitable pigments or dyes for seed treatment formulations are pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Stickers/adhesion agents are added to improve the adhesion of the active materials on the seeds after treatment. Suitable adhesives are block copolymers EO/PO surfactants but also polyvinylalcohols, polyvinylpyrrolidones, polyacrylates, polymethacrylates, polybutenes, polyisobutylenes, polystyrene, polyethyleneamines, polyethyleneamides, polyethyleneimines (Lupasol®, Polymin®), polyethers and copolymers derived from these polymers.

Compositions which are useful for seed treatment are e.g.:

A Soluble concentrates (SL, LS)

D Emulsions (EW, EO, ES)

E Suspensions (SC, OD, FS)

F Water-dispersible granules and water-soluble granules (WG, SG)

G Water-dispersible powders and water-soluble powders (WP, SP, WS)

H Dustable powders (DP, DS)

For use against ants, termites, wasps, flies, mosquitoes, crickets, or cockroaches, compounds of formula I are preferably used in a bait composition.

The bait can be a liquid, a solid or a semisolid preparation (e.g. a gel). Solid baits can be formed into various shapes and forms suitable to the respective application e.g. granules, blocks, sticks, disks. Liquid baits can be filled into various devices to ensure proper application, e.g. open containers, spray devices, droplet sources, or evaporation sources. Gels can be based on aqueous or oily matrices and can be formulated to particular necessities in terms of stickiness, moisture retention or aging characteristics.

The bait employed in the composition is a product which is sufficiently attractive to incite insects such as ants, termites, wasps, flies, mosquitoes, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

Formulations of compounds of formula I as aerosols (e.g. in spray cans), oil sprays or pump sprays are highly suitable for the non-professional user for controlling pests such as flies, fleas, ticks, mosquitoes or cockroaches. Aerosol recipes are preferably composed of the active compound, solvents such as lower alcohols (e.g. methanol, ethanol, propanol, butanol), ketones (e.g. acetone, methyl ethyl ketone), paraffin hydrocarbons (e.g. kerosenes) having boiling ranges of approximately 50 to 250° C., dimethylformamide, N-methylpyrrolidone, dimethyl sulfoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used.

The compounds of formula I and its respective compositions can also be used in mosquito and fumigating coils, smoke cartridges, vaporizer plates or long-term vaporizers and also in moth papers, moth pads or other heat-independent vaporizer systems.

Methods to control infectious diseases transmitted by insects (e.g. malaria, dengue and yellow fever, lymphatic, filariasis, and leishmaniasis) with compounds of formula I and its respective compositions also comprise treating surfaces of huts and houses, air spraying and impregnation of curtains, tents, clothing items, bed nets, tsetse-fly trap or the like. Insecticidal compositions for application to fibers, fabric, knitgoods, nonwovens, netting material or foils and tarpaulins preferably comprise a mixture including the insecticide, optionally a repellent and at least one binder. Suitable repellents for example are N,N-diethyl-meta-toluamide (DEET), N,N-diethylphenylacetamide (DEPA), 1-(3-cyclohexan-1-yl-carbonyl)-2-methylpiperine, (2-hydroxymethylcyclohexyl)acetic acid lactone, 2-ethyl-1,3-hexandiol, indalone, Methylneodecanamide (MNDA), a pyrethroid not used for insect control such as {(+/−)-3-allyl-2-methyl-4-oxocyclopent-2-(+)-enyl-(+)-trans-chrysantemate (Esbiothrin), a repellent derived from or identical with plant extracts like limonene, eugenol, (+)-Eucamalol (1), (−)-1-epi-eucamalol or crude plant extracts from plants like Eucalyptus maculata, Vitex rotundifolia, Cymbopogan martinii, Cymbopogan citratus (lemon grass), Cymopogan nartdus (citronella). Suitable binders are selected for example from polymers and copolymers of vinyl esters of aliphatic acids (such as such as vinyl acetate and vinyl versatate), acrylic and methacrylic esters of alcohols, such as butyl acrylate, 2-ethylhexylacrylate, and methyl acrylate, mono- and di-ethylenically unsaturated hydrocarbons, such as styrene, and aliphatic diens, such as butadiene.

The impregnation of curtains and bednets is mostly done by dipping the textile material into emulsions or dispersions of the insecticide or spraying them onto the nets.

The compounds of formula I and its compositions can be used for protecting non-living material, in particular cellulose-based materials such as wooden materials e.g. trees, board fences, sleepers, etc. and buildings such as houses, outhouses, factories, but also construction materials, furniture, leathers, fibers, vinyl articles, electric wires and cables etc. from ants and/or termites, and for controlling ants and termites from doing harm to crops or human being (e.g. when the pests invade into houses and public facilities). The compounds of formula I are applied not only to the surrounding soil surface or into the under-floor soil in order to protect wooden materials but it can also be applied to lumbered articles such as surfaces of the under-floor concrete, alcove posts, beams, plywoods, furniture, etc., wooden articles such as particle boards, half boards, etc. and vinyl articles such as coated electric wires, vinyl sheets, heat insulating material such as styrene foams, etc. In case of application against ants doing harm to crops or human beings, the ant controller of the present invention is applied to the crops or the surrounding soil, or is directly applied to the nest of ants or the like.

In the methods according to the invention the pests are controlled by contacting the target parasite/pest, its food supply, habitat, breeding ground or its locus with a pesticidally effective amount of compounds of formula I or with a salt thereof or with a composition, containing a pesticidally effective amount of a compound of formula I or a salt thereof.

“Locus” means a habitat, breeding ground, plant, seed, soil, area, material or environment in which a pest or parasite is growing or may grow.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

The compounds of the invention can also be applied preventively to places at which occurrence of the pests is expected.

The compounds of formula I may be also used to protect growing plants from attack or infestation by pests by contacting the plant with a pesticidally effective amount of compounds of formula I. As such, “contacting” includes both direct contact (applying the compounds/compositions directly on the pest and/or plant—typically to the foliage, stem or roots of the plant) and indirect contact (applying the compounds/compositions to the locus of the pest and/or plant).

In the case of soil treatment or of application to the pests dwelling place or nest, the quantity of active ingredient ranges from 0.0001 to 500 g per 100 m², preferably from 0.001 to 20 g per 100 m².

Customary application rates in the protection of materials are, for example, from 0.01 g to 1000 g of active compound per m² treated material, desirably from 0.1 g to 50 g per m².

Insecticidal compositions for use in the impregnation of materials typically contain from 0.001 to 95 weight %, preferably from 0.1 to 45 weight %, and more preferably from 1 to 25 weight % of at least one repellent and/or insecticide.

For use in bait compositions, the typical content of active ingredient is from 0.001 weight % to 15 weight %, desirably from 0.001 weight % to 5% weight % of active compound.

For use in spray compositions, the content of active ingredient is from 0.001 to 80 weights %, preferably from 0.01 to 50 weight % and most preferably from 0.01 to 15 weight %.

For use in treating crop plants, the rate of application of the active ingredients of this invention may be in the range of 0.1 g to 4000 g per hectare, desirably from 25 g to 600 g per hectare, more desirably from 50 g to 500 g per hectare.

In the treatment of seed, the application rates of the mixture are generally from 0.1 g to 10 kg per 100 kg of seed, preferably from 1 g to 5 kg per 100 kg of seed, in particular from 1 g to 200 g per 100 kg of seed.

The present invention is now illustrated in further detail by the following examples.

The products were characterized by coupled High Performance Liquid Chromatography/mass spectrometry (HPLC/MS), by NMR or by their melting points. HPLC column: RP-18 column (Chromolith Speed ROD from Merck KgaA, Germany). Elution: acetonitrile+0.1% trifluoroacetic acid (TFA)/water in a ratio of from 5:95 to 95:5 in 5 minutes at 40° C.

In the examples the following abbreviations are used:

m.p.: melting point

THF: tetrahydrofuran

MS: Quadrupol electrospray ionisation, 80 V (positiv modus)

### EXAMPLE 1

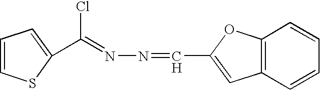

A mixture of 12 g (44.4 mmol) of thiophene-2-carboxylic acid [(benzofuran-2-yl)methylene]hydrazide, 29.1 g (110.9 mmol) of triphenylphosphine and 10.3 ml (16.4 g, 106.5 mmol) of carbon-tetrachloride in acetonitrile was stirred at room temperature overnight. After addition of water the desired product was extracted with 4×200 ml of hexane to yield 6.0 g (20.8 mmol, 47%) of the title compound which was analyzed by ¹H-NMR (m.p. 96-98° C.).

### EXAMPLE 2

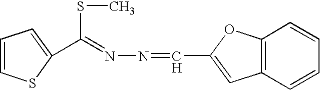

A mixture of 0.8 g (2.8 mmol) of the compound of example 1, 0.6 g (8.6 mmol) of sodium thiomethylate and 1.1 g (8.0 mmol) of potassium carbonate in 15 ml of THF was stirred at room temperature overnight. After evaporation of the solvent and addition of water the desired product was extracted with 2×40 ml of diethyl ether. 0.2 g (0.7 mmol, 25%) of the title compound were obtained which were analyzed by ¹H-NMR (m.p. 68-70° C.).

### EXAMPLE 3

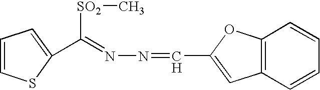

0.74 g (77% purity, 3.3 mmol) of m-chloroperbenzoic acid were added to 0.5 g (1.7 mmol) of the compound of example 2 in 20 ml of dichloromethane. The resulting solution was stirred at room temperature overnight and was subsequently washed with saturated sodium hydrogencarbonate solution. The crude product was purified by column chromatography (silica gel) using 50% diethyl ether in hexane to give 0.4 g (1.2 mmol, 71%) of the title compound which was analyzed by ¹H-NMR and MS (m.p. 105-110° C.).

### EXAMPLE 4

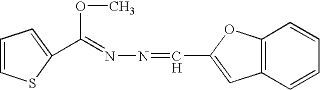

A mixture of 0.6 g (2.1 mmol) of the compound of example 1 and 15 ml of sodium methoxide in methanol (0.5 M) was stirred at room temperature overnight. After evaporation of the solvent the residue was dissolved in 20 ml of water and the product was extracted with 2×40 ml of diethyl ether to give 0.6 g (2.1 mmol, 1.00%) of the desired product which was analyzed by ¹H-NMR and GC-MS.

### EXAMPLE 5

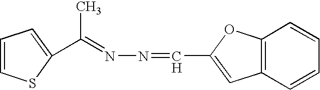

1.7 ml of a 3M solution of methyl magnesiumbromide in diethyl ether were added to 0.5 g (1.7 mmol) of the compound of example 1 in 5 ml THF. The resulting mixture was stirred at room temperature overnight. After quenching with water the organic solvent was evaporated and the desired product was extracted with 2×100 ml of diethyl ether to give 0.2 g (0.7 mmol, 41%) of the title compound.

The compounds of examples 6 to 10, which are shown in table B, were obtained by analogy.

TABLE B

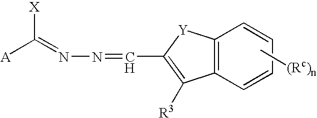

| Exam- | | | | A | | m.p. |
|---|---|---|---|---|---|---|
| ple | A | X | Y | R³ | (R^{c})ₙ | [° C.] |
| 6 | thien-2-yl | Cl | S | CH₃ | — | 163-165 |
| 7 | thien-2-yl | SO₂CH₃ | CH₂—CH₂ | H | — | 122-126 |
| 8 | thien-2-yl | SCH₃ | CH₂—CH₂ | H | — | — |
| 9 | pyrid-3-yl | Cl | O | H | — | — |
| 10 | pyrid-3-yl | OCH₃ | O | H | — | — |

### EXAMPLE 11

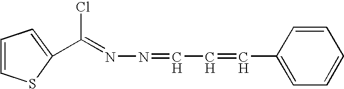

The title compound was obtained from thiophene-2-carboxylic acid [3-(phenyl)propen-1-ylidene]hydrazide by analogy to the process of example 1.

### EXAMPLE 12

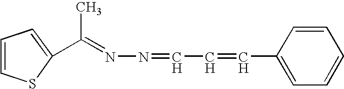

4 ml of a 3M solution of methyl magnesiumbromide in diethyl ether were added to 0.8 g (2.9 mmol) of the compound of example 11 in 5 ml of THF. The resulting mixture was stirred at room temperature overnight. After quenching with water the organic solvent was evaporated and the desired product was extracted twice with each 100 ml of diethyl ether. The crude product was purified by column chromatography (silica gel) using 10% diethyl ether in hexane to give 0.2 g (0.8 mmol, 28%) of the E-isomer of Example 12 which was analyzed by ¹H-NMR (m.p. 72-74° C.). The Z-isomer was also obtained but was not analytically pure.

The compounds of examples 13 to 85, which are shown in table C, were obtained by analogy.

TABLE C

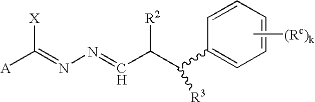

| | | | | | | m.p. or |
|---|---|---|---|---|---|---|
| Exp. | A | X | R² | R³ | (R^{c})ₖ | RT [min], m/z |
| 13 | thien-2-yl | SO₂CH₃ | CH₃ | H | — | 103-107° C. |
| 14 | thien-2-yl | Cl | CH₃ | H | 4-F | 86-89° C. |
| 15 | thien-2-yl | SCH₂CF₃ | CH₃ | H | — | 88-90° C. |
| 16 | thien-2-yl | SCH₃ | CH₃ | H | 4-F | 63-68° C. |
| 17 | thien-2-yl | Cl | CH₃ | H | — | 85-87° C. |
| 18 | thien-2-yl | Cl | F | H | — | 84-86° C. |
| 19 | thien-2-yl | Cl | Cl | H | — | 4.457, |
| | | | | | | 309 [M + H]⁺ |
| 20 | 4,5-dibromothien- | Cl | CH₃ | H | — | 90-91° C. |
| | 2-yl | | | | | |
| 21 | 3-chloro-4- Cl | CH₃ | H | — | 85-86° C. | |
| | methylthien-2-yl | | | | | |
| 22 | 3-chloro-4- | Cl | H | H | — | 78-82° C. |
| | methylthien-2-yl | | | | | |
| 23 | 3-methyl-thien- | Cl | CH₃ | H | — | 62-64° C. |
| | 2-yl | | | | | |
| 24 | 5-methyl-thien- | SCH₃ | CH₃ | H | — | 63-67° C. |
| | 2-yl | | | | | |
| 25 | 5-methyl-thien- | SCH₂CF₃ | CH₃ | H | — | 85-87° C. |
| | 2-yl | | | | | |
| 26 | pyrid-2-yl | Cl | CH₃ | H | — | 3.858, |
| | | | | | | 384 [M + H]⁺ |
| 27 | pyrid-2-yl | OCH₃ | CH₃ | H | — | |
| 28 | 6-chloropyrid-3-yl | Cl | CH₃ | H | — | 91-93° C. |
| 29 | 6-chloropyrid-4-yl | Cl | CH₃ | H | — | 52-53° C. |
| 30 | 6-chloropyrid-4-yl | Cl | H | H | — | 95° C. |
| 31 | 2-fluorophenyl | Cl | CH₃ | H | 3-F | 4.534, |
| | | | | | | 319 [M + H]⁺ |
| 32 | 2-fluorophenyl | OCH₃ | CH₃ | H | 3-F | |
| 33 | thien-2-yl | Cl | CH₃ | H | 3-OCHF₂ | |
| 34 | thien-2-yl | Cl | CH₃ | H | 3-CF₃ | |
| 35 | thien-2-yl | Cl | CH₃ | H | 3-CF₃, 4-F | |
| 36 | thien-2-yl | OCH₃ | CH₃ | H | 3-CF₃ | |
| 37 | thien-2-yl | OCH₂CH₃ | CH₃ | H | — | |
| 38 | thien-2-yl | OCH₃ | CH₃ | H | — | |
| 39 | thien-2-yl | OCH₃ | CH₃ | H | 4-F, 3-CF₃ | |
| 40 | thien-2-yl | OCH₃ | CH₃ | H | 4-OCH₃, 3-CF₃ | |
| 41 | thien-2-yl | E—SCH(CH₃)₂ | CH₃ | H | — | |
| 42 | thien-2-yl | SCH₃ | H | H | — | |
| 43 | thien-2-yl | SC(CH₃)₃ | CH₃ | H | — | |
| 44 | thien-2-yl | Z—SC(CH₃)₃ | CH₃ | H | — | |
| 45 | thien-2-yl | S—C(O)CH₃ | CH₃ | H | — | |
| 46 | thien-2-yl | SCH₃ | CH₃ | H | 3-OCHF₂ | |
| 47 | thien-2-yl | Z—SCH₃ | CH₃ | H | — | |
| 48 | thien-2-yl | E—SCH₃ | CH₃ | H | — | |
| 49 | thien-2-yl | Z—SCH₂CH₃ | CH₃ | H | — | |
| 50 | thien-2-yl | E—SCH₂CH₃ | CH₃ | H | — | |
| 51 | thien-2-yl | Z—SCH₂CH₃ | H | H | — | |
| 52 | thien-2-yl | E—SCH₂CH₃ | H | H | — | |
| 53 | thien-2-yl | SCH₃ | F | H | — | |
| 54 | thien-2-yl | SCH₃ | CH₃ | H | 3-CF₃ | |
| 55 | thien-2-yl | SCH₃ | CH₃ | H | 4-SCH₃, 3-CF₃ | |
| 56 | thien-2-yl | Z—CH₃ | CH₃ | H | — | |
| 57 | thien-2-yl | E—CH₃ | CH₃ | H | — | |
| 58 | thien-2-yl | CH₂CH₃ | CH₃ | H | — | |
| 59 | thien-2-yl | CH₃ | F | H | — | |
| 60 | 5-chlorothien-2-yl | OCH₃ | CH₃ | H | — | |
| 61 | 5-chlorothien-2-yl | Cl | CH₃ | H | — | |
| 62 | 5-chlorothien-2-yl | SCH₃ | CH₃ | H | — | |
| 63 | 5-methyl-thien-2- | Cl | CH₃ | H | — | |
| | yl | | | | | |
| 64 | 5-methyl-thien-2- | OCH₃ | CH₃ | H | — | |
| | yl | | | | | |
| 65 | 5-methyl-thien-2- | Z—SCH₂CH₃ | CH₃ | H | — | |
| | yl | | | | | |
| 66 | 5-methyl-thien-2- | E—SCH₂CH₃ | CH₃ | H | — | |
| | yl | | | | | |
| 67 | 5-methyl-thien-2- | Z— | CH₃ | H | — | |
| | yl | SCH₂CH₂CH₃ | | | | |
| 68 | 5-methyl-thien-2- | E— | CH₃ | H | — | |
| | yl | SCH₂CH₂CH₃ | | | | |
| 69 | 3-methyl-thien-2- | OCH₃ | CH₃ | H | — | |
| | yl | | | | | |
| 70 | 3-methyl-thien-2- | Z—SCH₂CF₃ | CH₃ | H | — | |
| | yl | | | | | |
| 71 | 3-methyl-thien-2- | E—SCH₂CF₃ | CH₃ | H | — | |
| | yl | | | | | |
| 72 | 3-methyl-thien-2- | E—SCH₂CH₃ | CH₃ | H | — | |
| | yl | | | | | |
| 73 | 3-methyl-thien-2- | Z—SCH₂CH₃ | CH₃ | H | — | |
| | yl | | | | | |
| 74 | 6-methylthio- | Z—SCH₃ | CH₃ | H | — | |
| | pyrid-3-yl | | | | | |
| 75 | 6-methylthio- | E—SCH₃ | CH₃ | H | — | |
| | pyrid-3-yl | | | | | |
| 76 | 6-chloropyrid-3-yl | OCH₃ | CH₃ | H | — | |
| 77 | pyrid-3-yl | OCH₃ | CH₃ | H | — | |
| 78 | pyrid-3-yl | Cl | CH₃ | H | 3-CF₃ | |
| 79 | pyrid-3-yl | Cl | CH₃ | H | — | |
| 80 | pyrid-3-yl | Z—SCH₃ | CH₃ | H | — | |
| 81 | pyrid-3-yl | E—SCH₃ | CH₃ | H | — | |
| 82 | pyrid-3-yl | Cl | CH₃ | H | 4-F, 3-CF₃ | |
| 83 | pyrid-3-yl | SCH₃ | CH₃ | H | 4-SCH₃, 3-CF₃ | |
| 84 | pyrid-3-yl | OCH₃ | CH₃ | H | 3-CF₃ | |
| 85 | thien-2-yl-methyl | Cl | CH₃ | H | — | |

The action of the compounds of the formula I against pests was demonstrated by the following experiments:

I. Nematicidal Evaluation

Test compounds were prepared and formulated into aqueous formulations using acetone. The formulations were tested using root knot nematode (2nd instar) and soybean cyst nematode (2nd instar) as target species.
   I.1 Test Procedures for Root-Knot Nematode (Meloidogyne incognita):
Tomato plants (var. Bonny Best) were grown in the greenhouse in plastic tubs (4 to 6 plants per tub). The plants and soil (a 50:50 mixture of sand and “New Egypt” sandy loam) were infested with M. incognita J2 (to establish the “in-house” colony, M. incognita J2 were initially acquired from Auburn University). The plants were kept pruned and were used on an “as needed” basis. The tomato plants were kept in the cylinder containing hydroponic solution and aerated until the nematodes were no longer present in the solution (usually about 60 days). The cultures were checked daily by eluting a small volume (approximately 20 ml) from the bottom of a funnel attached to the cylinder into a small crystallizing dish and observed using a binocular dissecting scope. If needed for testing, the nematodes were cleaned and concentrated by pouring the culture solution through a sieve for cleaning and a sieve for concentrating. The nematodes were then resuspended in water to a concentration of approximately 20 to 50 nematodes per 50 μl. These were counted by putting 25 μl of the nematode solution into a well of an unused well of an assay plate. The total was then multiplied by 2 for a final total of nematodes per 50 μl of solution. To microtiter plates containing about 1.0 mg of compound, 80:20 acetone was added to each well and the solution was mixed to obtain the desired compound concentration. The nematode solution was added to each plate. The plates were then sealed and they were placed in an incubator at 27° C. and 50% (+/−2%) relative humidity. After 72 hours, the population mortality was read, whereby immobility of nematodes was regarded as mortality.

In this test, compounds of examples 3, 4, 6, 8, 12, 14, 54, 56, 57, 70, 71 and 73 at 100 ppm showed over 95% mortality compared to untreated controls.

I.2 Test Procedures for Soybean Cyst Nematode (Heterodera Glycine):

The soybean bean cyst nematode culture was maintained in a greenhouse and soybean eggs and J2 larvae were obtained for testing by dislodging soybean cysts from the roots with a sieve. The cysts were broken to release the eggs and the eggs were maintained in water. The eggs hatched after 5-7 days at 28° C. To microtiter plates containing about 150 μg of compound, 80:20 acetone was added to each well and the solution was mixed to obtain the desired compound concentration. The nematode solution was added to the plate. The plates were then sealed and placed in an incubator at 27° C. and 50% (+/−2%) relative humidity. After 72 hours, the population mortality was read, whereby immobility of nematodes was regarded as mortality.
In this test, compounds of examples 3, 8, 11, 34, 52, 54 and 57 at 100 ppm showed over 95% mortality compared to untreated controls.
   II Activity Against Insects
   II.1 Cotton Aphid (Aphis Gossypii)
The active compounds were formulated in 50:50 acetone:water and 100 ppm Kinetics surfactant.
Cotton plants at the cotyledon stage (one plant per pot) were infested by placing a heavily infested leaf from the main colony on top of each cotyledon. The aphids were allowed to transfer to the host plant overnight, and the leaf used to transfer the aphids was removed. The cotyledons were dipped in the test solution and allowed to dry. After 5 days, mortality counts were made.
In this test, compounds of examples 2, 10, 24, 30, 33, 35, 42, 43, 46, 47, 48, 49, 50, 51, 65, 72, 78 and 84 at 300 ppm showed over 75% mortality in comparison with untreated controls.
   II.2 Green Peach Aphid (Myzus Persicae)
The active compounds were formulated in 50:50 acetone:water and 100 ppm Kinetic® surfactant.
Pepper plants in the 2nd leaf-pair stage (variety ‘California Wonder’) were infested with approximately 40 laboratory-reared aphids by placing infested leaf sections on top of the test plants. The leaf sections were removed after 24 hr. The leaves of the intact plants were dipped into gradient solutions of the test compound and allowed to dry. Test plants were maintained under fluorescent light (24 hour photoperiod) at about 25° C. and 20-40% relative humidity. Aphid mortality on the treated plants, relative to mortality on check plants, was determined after 5 days.
In this test, compounds of examples 1, 5, 12, 14, 17, 22, 28, 37, 38, 59, 61, 63, 69, 79, 80, 81 and 85 at 300 ppm showed over 75% mortality in comparison with untreated controls.
   II.3 Bean Aphid (Aphis Fabae)
The active compounds were formulated in 50:50 acetone:water and 100 ppm Kinetics surfactant.
Nasturtium plants grown in Metro mix in the 1^{st} leaf-pair stage (variety ‘Mixed Jewle’) were infested with approximately 2-30 laboratory-reared aphids by placing infested cut plants on top of the test plants. The cut plants were removed after 24 hr. Each plant was dipped into the test solution to provide complete coverage of the foliage, stem, protruding seed surface and surrounding cube surface and allowed to dry in the fume hood. The treated plants were kept at about 25° C. with continuous fluorescent light. Aphid mortality was determined after 3 days.
In this test, compounds of examples 9 and 79 at 300 ppm showed over 70% mortality in comparison with untreated controls.
   II.4 Silverleaf Whitefly (Bemisia Argentifolii)
The active compounds were formulated in 50:50 acetone:water and 100 ppm Kinetics surfactant.
Selected cotton plants were grown to the cotyledon state (one plant per pot). The cotyledons were dipped into the test solution to provide complete coverage of the foliage and placed in a well-vented area to dry. Each pot with treated seedling was placed in a plastic cup and 10 to 12 whitefly adults (approximately 3-5 day old) were introduced. The insects were collected using an aspirator and an 0.6 cm, non-toxic Tygon® tubing (R-3603) connected to a barrier pipette-tip. The tip, containing the collected insects, was then gently inserted into the soil containing the treated plant, allowing Insects to crawl out of the tip to reach the foliage for feeding. The cups were covered with a re-usable screened lid (150 micron mesh polyester screen PeCap from Tetko Inc). Test plants were maintained in the holding room at about 25° C. and 20-40% relative humidity for 3 days avoiding direct exposure to the fluorescent light (24 hour photoperiod) to prevent trapping of heat inside the cup. Mortality was assessed 3 days after treatment of the plants.
In this test, compounds of examples 16, 57 and 77 at 300 ppm showed over 70% mortality in comparison with untreated controls.
   II.5 Orchid Thrips (Dichromothrips Corbetti)
Dichromothrips corbetti adults used for bioassay were obtained from a colony maintained continuously under laboratory conditions. For testing purposes, the test compound was diluted to a concentration of 500 ppm (wt compound: vol diluent) in a 1:1 mixture of acetone:water, plus 0.01% Kinetics surfactant.
Thrips potency of each compound was evaluated by using a floral-immersion technique. Plastic petri dishes were used as test arenas. All petals of individual, intact orchid flowers were dipped into treatment solution for approximately 3 seconds and allowed to dry for 2 hours. Treated flowers were placed into Individual petri dishes along with 10-15 adult thrips. The petri dishes were then covered with lids. All test arenas were held under continuous light and a temperature of about 28° C. for duration of the assay. After 4 days, the numbers of live thrips were counted on each flower, and along inner walls of each petri dish. The level of thrips mortality was extrapolated from pre-treatment thrips numbers.
In this test, compounds of examples 13, 29, 56, 64 and 69 at 500 ppm showed over 95% mortality in comparison with untreated controls.

We claim:

1. An azine compound of the general formula I 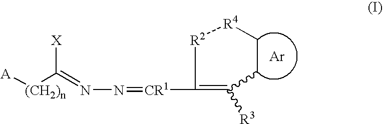
and the salts thereof, wherein
. . . is absent or a covalent bond;
n is 0 or 1;
A is a cyclic radical selected from phenyl and a 5- or 6-membered heterocyclic radical with 1, 2, 3 or 4 heteroatoms which are selected, independently of one another, from O, N and S, and where the cyclic radical may have 1, 2, 3, 4 or 5 substituents R^{a} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁵R⁵, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, R⁵R⁶N—CO—, phenyl, benzyl and phenoxy, wherein phenyl, benzyl and phenoxy may be substituted by 1, 2, 3, 4 or 5 substituents R^{b} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁵R⁶, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl and R⁵R⁶N—CO—;
Ar is an aromatic radical selected from phenyl, pyridyl, pyrimidyl furyl and thienyl, where the aromatic radical may carry 1 to 5 substituents R^{c} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, R⁵R⁶N—CO—, phenyl and phenoxy, wherein phenyl and phenoxy may be unsubstituted or substituted by 1, 2, 3, 4 or 5 substituents R^{b} as defined above;
X is selected from halogen, OR⁷, SR⁷, SOR⁷, SO₂R⁷, C₁-C₄-alkyl and C₁-C₄-haloalkyl;
R¹ is H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₂-C₁₀-haloalkynyl or phenyl which may be substituted by 1, 2, 3, 4 or 5 substituents R^{d} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁵R⁶, C₁-C₁₀-alkoxycarbonyl, C₁-C₁₀-haloalkoxycarbonyl, C₁-C₁₀-alkenyloxycarbonyl, C₂-C₁₀-haloalkenyloxycarbonyl, C₁-C₁₀-alkylcarbonyl, C₁-C₁₀-haloalkylcarbonyl and R⁵R⁶N—CO—;
R² is a monovalent radical selected from H, halogen, cyano, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl and phenyl which may be substituted by 1, 2, 3, 4 or 5 substituents R^{b} as defined above;
R³ is H, halogen, cyano, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl or phenyl which may be substituted by 1 to 5 substituents R^{b} as defined above;
R⁴ is hydrogen or has one of the meanings given for R^{c} or
R⁴ together with R² is a bivalent radical, which is selected from O, S, N—R⁸, CR⁹═N, CH₂—CH₂, O—C(O) or OCH₂;
R⁵ and R⁶, independently of one another, are H or C₁-C₁₀-alkyl;
R⁷ is selected from C₁-C₁₀-alkyl, C₁-C₁₀-alkylcarbonyl, C₁-C₁₀-haloalkyl, C₂-C₁₀-alkenyl, C₂-C₁₀-haloalkenyl, C₃-C₁₀-cycloalkyl and C₃-C₁₀-halocycloalkyl;
R⁸ is hydrogen, cyano, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-haloalkylsulfonyl, C₁-C₁₀-alkylcarbonyl, C₁-C₁₀-haloalkylcarbonyl, R⁵R⁶N—CO—, phenyl or benzyl, wherein phenyl and benzyl may be substituted by 1, 2, 3, 4 or 5 substituents R^{b}; and
R⁹ is hydrogen or has one of the meanings given for R^{c};

2. The compound as claimed in claim 1, wherein Ar in formula I is phenyl, which is unsubstituted or substituted by 1, 2, 3 or 4 radicals R^{c} as defined above.

3. The compound as claimed in claim 1, wherein A in formula I is a cyclic radical selected from phenyl, thienyl, furyl, pyrrolyl, pyrazolyl, imidazolyl, oxazolyl, isoxazolyl, thiazolyl, isothiazoloyl, pyridyl, pyrimidinyl, pyrazinyl, and pyridazinyl and where the cyclic radical may be substituted by 1, 2 or 3 substituents R^{a} as defined above.

4. The compound as claimed in claim 1, wherein A in formula I is a cyclic radical selected from phenyl, thienyl, and pyridyl, where the cyclic radical may be substituted by 1, 2 or 3 substituents R^{a} which are selected, independently of one another, from halogen, C₁-C₄-alkyl, C₁-C₄-alkoxy, C₁-C₄-haloalkoxy and C₁-C₄-haloalkyl.

5. The compound as claimed in claim 1, wherein A in formula I is 2-thienyl which may be substituted by 1, 2 or 3 substituents R^{a} which are selected, independently of one another, from halogen, C₁-C₄-alkyl, C₁-C₄-alkoxy, C₁-C₄-haloalkoxy and C₁-C₄-haloalkyl.

6. The compound as claimed in claim 1, wherein X in formula I is selected from Cl, Br, OR⁷, SR⁷, SO₂R⁷ and methyl, wherein R⁷ is selected from C₁-C₄-alkyl and C₁-C₂-fluoroalkyl.

7. The compound as claimed in claim 1, wherein X in formula I is selected from Cl, OCH₃, OCHF₂, SCH₃, SO₂CH₃, SO₂CF₃, SO₂CH₂CF₃ and SCF₃.

8. The compound of the general formula I as defined in claim 1, wherein R¹ is hydrogen.

9. The compound as claimed in claim 1, wherein R² in formula I is selected from hydrogen, halogen, C₁-C₄-alkyl, C₁-C₄-alkoxy, C₁-C₄-haloalkoxy and C₁-C₄-haloalkyl.

10. The compound as claimed in claim 1, wherein R³ in formula I is selected from hydrogen, halogen and C₁-C₄-alkyl.

11. The compound as claimed in claim 1, wherein R³ in formula I is selected from hydrogen, fluorine, chlorine and methyl.

12. The compound as claimed in claim 1, wherein R⁴ is hydrogen or a radical R^{c} as defined above.

13. The compound as claimed in claim 12, wherein Ar is phenyl, which is unsubstituted or substituted by 1, 2 or 3 radicals R^{c} which are selected, independently of one another, from halogen, CN, C₁-C₄-alkyl, C₁-C₄-alkoxy, C₁-C₄-haloalkoxy and C₁-C₄haloalkyl.

14. The compound as claimed in claim 1, wherein R⁴ together with R² is a bivalent radical, which is selected from 0 and O—C(O).

15. The compound as claimed in claim 4 wherein Ar in formula I is phenyl, which is unsubstituted or which may carry 1, 2 or 3 radicals R^{c} which are selected, independently of one another, from halogen, CN, C₁-C₄-alkyl, C₁-C₄-alkoxy, C₁-C₄-haloalkoxy and C₁-C₄-haloalkyl.

16. A composition for combating pests, selected from insects, arachnids and nematodes, which comprises a pesticidally effective amount of at least one compound of the general formula I as defined in claim 1 or a salt thereof and at least one Inert carrier and/or at least one surfactant.

17. A method for combating pests, selected from insects, arachnids and nematodes, which comprises contacting said pests, their habitat, breeding ground, food supply, plant, seed, soil, area, material or environment in which the animal pests are growing or may grow, or the materials, plants, seeds, soils, surfaces or spaces to be protected from an attack of or infestation by said pest, with a pesticidally effective amount of a compound of the general formula I as defined in claim 1 or a salt thereof.

18. The method as claimed in claim 17, wherein the pests are insects.

19. The method as claimed in claim 17, wherein the pests are nematodes.

20. A method for protecting crops from attack or infestation by pests, selected from insects, arachnids and nematodes, the method comprising contacting a crop with a pesticidally effective amount of at least one compound of formula I as defined in claim 1 or with a salt thereof.

21. A method for protecting non-living materials from attack or infestation by pests, selected from insects, arachnids and nematodes, the method comprising contacting the non-living material with a pesticidally effective amount of at least one compound of formula I as defined in claim 1 or with a salt thereof.
